(19) United States
(12) Patent Application Publication
(10) Pub. No.: US 2021/0077304 A1
(43) Pub. Date: Mar. 18, 2021

(54) FUNCTIONAL FABRIC DEVICES HAVING INTEGRATED SENSORS

(71) Applicant: NORTHWESTERN UNIVERSITY, Evansto, IL (US)

(72) Inventors: Shuai XU, Chicago, IL (US); In Hwa JUNG, Evanston, IL (US); John A. ROGERS, Evanston, IL (US)

(21) Appl. No.: 16/611,923

(22) PCT Filed: May 10, 2018
(86) PCT No.: PCT/US2018/032082

§ 371 Date: Nov. 8, 2019

Related U.S. Application Data
(60) Provisional application No. 62/504,271, filed on May 10, 2017.

(51) Int. Cl. A61F 13/08; A61F 5/01; A41D 1/00; A61B 5/00; A61B 5/11; A61B 5/022

(52) U.S. Cl. CPC A61F 13/08; A61B 2562/0247; A41D 1/002; A61B 5/6804; A61B 5/0008; A61B 5/002; A61B 5/1117; A61B 5/022; A61B 5/447; A61B 5/6832; A61F 2005/0188; A41D 2500/10; A41D 2500/20; A41D 2500/30; A61B 2562/0219; A61B 2562/0271; A61B 2562/029; A61F 5/01

(57) ABSTRACT

Provided herein are smart functional fabrics and therapeutic/diagnostic garments which utilize flexible wireless electronic devices to enhance functionality, for example, by measuring key therapy parameters and providing data to clinicians, and methods utilizing such devices. The provided methods are designed to avoid patient discomfort and decrease harm or irritation caused by garments which utilize bulkier, more rigid sensors. Additionally, the described devices are multiplexed to allow for sensing of multiple parameters of therapeutic interest and combinations of measured data for new clinical metrics.

### CROSS-REFERENCE TO RELATED APPLICATIONS

This application claims the benefit of priority to U.S. Provisional Patent Application No. 62/504,271 filed on May 10, 2017, which is specifically incorporated by reference to the extent not inconsistent herewith.

### BACKGROUND OF INVENTION

Functional fabrics (e.g. therapeutic or diagnostic garments which provide compression, heat, cooling, monitoring, etc. for the treatment and/or monitoring of a condition) have received increased interest over the past several years due to their ability to treat serious ailments with minimal invasiveness and discomfort to a patient. For example, compression garments have been used to treat a variety of skin or blood flow ailments including edema, venous status ulcers, and lipodermatosclerosis.

Compression stockings for the treatment of skin and blood flow ailments in a patient's feet and legs have developed into a multibillion dollar per year industry. While current methods of treatment are often effective, they face significant challenges. For example, while compression stockings are rated by the manufacturer to provide a certain pressure, these ratings do not account for a patient's leg shape or size and may provide a different treatment pressure to each individual patient. Further, there are no mechanisms to determine when the fabric or material of the compression stocking have worn or deteriorated to the point they are no longer providing therapeutic levels of compression.

Other groups have begun to address problems with compression garments by creating smart therapeutic garments containing sensors and communication devices to monitor the actual therapy provided to a patient by a garment. For example, International Patent Publication No. 2016/073777 is directed towards telemedical wearable sensing systems for the management of chronic venous disorders. However, the simple inclusion of sensor and communication devices into therapeutic garments is problematic as the devices can cause patient discomfort, skin irritation, inflammation or, in some cases, negatively affect treatment by interfering with the desired therapy. For example, the mere presence, location and/or device configuration can significantly reduce or alter the provided compression, including in an uncontrolled manner.

It can be seen from the foregoing that there remains a need in the art for smart therapeutic garments utilizing advanced sensing and communicating devices with decreased bulk and rigidity to reduce patient discomfort and increase therapeutic value. Further, multiplexed wireless sensors allow for monitoring of both therapeutic efficacy and patient adherence, as well as new therapeutic metrics which provide medical professionals with advanced insight and understanding therapeutic garment treatments.

### SUMMARY OF THE INVENTION

Provided herein are functional fabrics, including therapeutic, diagnostic, and/or monitoring garments, which utilize flexible wireless electronic devices to enhance functionality, for example, by measuring key therapeutic parameters and providing data to clinicians and wearers, and methods utilizing such devices, including in a remote and wireless manner. The provided systems are designed to minimize patient discomfort and decrease harm or irritation caused by garments which utilize bulkier, more rigid sensors. Additionally, any of the described devices may be optionally multiplexed to facilitate sensing of multiple parameters of therapeutic interest and combinations of measured data for new clinical metrics. In this manner, a wearer has a much higher likelihood to conform to a recommended therapy as the systems and devices are so unobtrusive and comfortable, and as desired information such as a therapeutically relevant parameter may be remotely read, stored, monitored and/or reviewed, including by a medical caregiver or interested party. As desired, any of the functional fabrics may include one or more actuators that can exert a physical effect that can be either detected by a user (e.g., a warning signal), or to provide a biological benefit to the user (e.g., temperature regulation, pressure change, etc.).

In an aspect, provided is a system comprising: i) a functional fabric; and at least one flexible wireless device comprising: a) a pressure sensor; b) a wireless communication system; and c) a sensor for measurement of limb volume; wherein the at least one flexible wireless device is positioned in mechanical communication with a surface of the functional fabric. In an embodiment, for example, the multiplexed sensing of both limb volume and pressure allows targeted treatment of edema due to optimization of pressure applied and resultant reduction in limb volume.

In an aspect, provided is a system comprising: i) a functional fabric; and at least one flexible wireless device comprising: a) a pressure sensor; and b) a wireless communication system; wherein the at least one flexible wireless device is positioned in mechanical communication with a surface of the functional fabric; and wherein the flexible wireless device is wirelessly powered. In an embodiment, for example, wireless power allows the flexible device to operate without a battery, allowing for additional flexibility, reduced size (thickness or surface area) or both.

In an aspect, provided is a system comprising: i) a functional fabric; and at least one flexible wireless device comprising: a) a pressure sensor; and b) a wireless communication system; wherein the at least one flexible wireless device is positioned in mechanical communication with a surface of the functional fabric; wherein the flexible wireless device has dimensions and physical properties providing for conformal integration characterized by a contact stress of less than or equal to 40 kPa on a region of a body of a subject contacted with the system. In an embodiment, for example, the contact stress at the edges or interface between the flexible wireless device and the skin is less than or equal to 40 kPa. “Contact stress” can be used interchangeably with contact force, as they are mathematically related by the formula” Contact force=Contact pressure (e.g., contact stress) divided by contact area.

In an aspect, provided is a system comprising: i) a functional fabric; and at least one flexible wireless device comprising: a) a pressure sensor for measuring one or more pressures exerted by the functional fabric on a region of a body of a subject contacted with the functional fabric system; and b) a wireless communication system; wherein the at least one flexible device is positioned in mechanical communication with a surface of the functional fabric; wherein the flexible wireless device has dimensions and physical properties such that the pressure sensor affects the magnitude of the one or more pressures exerted by the functional fabric by less than or equal to a factor of 25%. In embodiments, for example, the magnitude of intramuscular pressure is reduced by less than 25% due to the presence of the flexible electronic device.

In an aspect, provided is a system comprising: i) a functional fabric; and at least one flexible wireless device comprising: a) a pressure sensor; and b) a wireless communication system; wherein the at least one flexible wireless device is positioned in mechanical communication with a surface of the functional fabric and the flexible wireless device; wherein the at least one flexible wireless device has dimensions and physical properties providing for conformal integration without substantial inflammation, irritation or discomfort of a region of a body of a subject contacted with the system. In embodiment, for example, patients do not feel or are indifferent to the presence of the flexible wireless device.

In an aspect, provided is a system comprising: i) a functional fabric providing at least one therapeutic or diagnostic benefit; ii) at least one flexible wireless device comprising: a sensor corresponding the said at least one therapeutic or diagnostic benefit of said functional fabric; a wireless communication system; and wherein said at least one flexible wireless device is positioned in mechanical communication with a surface of said functional fabric. For example, a functional fabric designed to provide compression therapy or pressure may have a corresponding pressure sensor, a functional fabric designed to heat or cool may have a temperature sensor, a functional fabric designed to add or reduce moisture may have a microfluidic sensor, a functional fabric designed to provide protection from ultraviolet light may have a UV sensor or a sunburn sensor.

The described systems may refer to any type of functional fabric or garment including, for example, a therapeutic garment, a diagnostic garment, a medical garment, a compression garment, an inelastic functional fabric, an elastic functional fabric, a woven functional fabric, a non-woven functional fabric or a knit functional fabric. Garment may be a sock, stocking, sleeve, glove, shirt, tights, skull cap, or other type of clothing. Garment may also refer to wraps, bandages, wound dressings, braces, hard casts, soft casts, splints, pneumatic compression devices and other medical devices. A functional fabric as described herein may be a therapeutic compression stocking. The functional fabric may be moistened, heated or cooled.

The systems and methods described herein use flexible wireless devices specifically designed to mimic skin properties such as strength, modulus and flexibility in order to minimize strain or discomfort of the skin. This promotes patient adherence as patients are more likely to continue a treatment if it does not irritate or cause discomfort. Systems having mechanical properties matched to underlying skin, such as elasticity, flexibility, and the like are more comfortable than systems that are not mechanically matched. The matching may be at a defined cutoff value, such as within a factor of 20, 10, 5 or 2. It also increases sensing accuracy as the sensor is closer to the surface of the skin and less likely to be separated from the skin surface.

The flexible wireless device may be affixed to the functional fabric via an adhesive. The flexible wireless device may be directly or indirectly supported by a region of a body of a subject contacted the system as described herein, for example, supported by the skin of the subject or supported by a fabric or other layer provided on the skin of the subject. The flexible wireless device may be characterized by an average Young's modulus less than or equal to 2 MPa, less than or equal to 1 MPa, or optionally, less than or equal to 0.75 MPa. The flexible wireless device may match the Young's modulus of a specific patient's skin, for example, being characterized by an average Young's modulus matched to within a factor of 20, to a factor of 10 or to a factor of 2 of the Young's modulus of the underlying skin of a subject, including in terms of an average or bulk modulus.

The flexible wireless device may have a net bending stiffness low enough to establish conformal contact with the skin of a subject, for example, a net bending stiffness of less than or equal to 2 nN m, less than or equal to 1 n Nm or less than or equal to 0.75 nN m. The flexible wireless device may have a net flexural rigidity of less than or equal to 2×10⁻⁴ Nm, less than or equal to 1×10⁻⁴ Nm, or optionally, less than or equal to 0.75×10⁻⁴ Nm.

The flexible wireless device may be made of more than one flexible or inflexible components such as sensors, substrates, electrical interconnects or conduits, circuits or other electronic components such that the combined properties of the wireless device provides the conformal contact or physical properties as described herein. The flexible wireless device may further comprise: a) a flexible substrate; b) a flexible electronic circuit supported by the flexible substrate, wherein the flexible electronic circuit comprises the pressure sensor and the wireless communication system; and c) a flexible superstrate layer encapsulating at least a portion of the flexible electronic circuit, the flexible substrate, or both the flexible electronic circuit and the flexible substrate. The flexible wireless substrate, the flexible wireless superstrate, the flexible wireless circuit or any combination thereof may be characterized by an average Young's modulus less than or equal to 2 MPa, less than or equal to 1 MPa, or optionally, less than or equal to 0.75 MPa. The flexible substrate and superstrate may encapsulate the electronic components of the device.

The flexible wireless device may be a stretchable device, for example, a stretchable device capable of undergoing elongation or compression to an extent of at least a factor of 1.2, a factor of 1.3, or optionally, a factor of 1.5 without system degradation or mechanical failure. The flexible substrate, flexible superstrate and/or flexible electronic components may also be stretchable. The stretchable device may further comprise one or more stretchable filamentary electrical interconnects. The stretchable filamentary electrical interconnects may have a serpentine, bent, folded, wavy or curved geometry.

The flexible wireless devices described herein may have one or more sensors to measure pressure, temperature, bioimpedence, radius of curvature, volume, contact (e.g. skin contact) or other physical properties of a patient. The flexible wireless device may have microfluidic sensors to measure sweat properties, for example, pH, salinity, etc. The pressure sensor may be a capacitance pressure sensor, piezoresistive pressure sensor, a liquid metal sensor or a combination thereof. The pressure sensor may measure instantaneous pressure, average pressure, cumulative pressure or any combination of these. The pressure sensor may measure the inherent extensibility of the functional fabric.

The flexible wireless devices may be able to measure properties of the garment being applied to the patient, for example, to measure treatment efficacy or degradation of the garment. The flexible wireless device may be capable of determining the static stiffness index of the functional fabric. The flexible wireless device may comprise one or more sensors selected from the group consisting of: a temperature sensor; a bioimpedence sensor; a radius of curvature sensor, an accelerometer, an heart rate sensor, a blood flow sensor, electrocardiography sensor, electromyography sensor, electroencephalography sensors, electrophysiological sensors, moisture sensor, humidity sensor, transcutaneous oxygen sensor, local blood flow sensor, and local redness sensor.

The flexible wireless device may further comprise a temperature sensor for measuring measures local temperature. The flexible wireless device may use the local temperature to provide a pressure measurement normalized by in situ temperature. The flexible wireless device may further comprise an accelerometer for monitoring the motion or movement of a subject or detecting the fall of a subject. The flexible wireless device may further comprise a wound healing sensor, for example for monitoring transcutaneous oxygen, local blood flow, local redness, local temperature or any combination of these.

The flexible wireless device may provide feedback to a user via various electronic means such as a visual display or haptic notification to provide a user feedback on treatment, time or efficacy of the device or functional fabric, for example, alerting the user to remove or replace the functional fabric. The flexible wireless device may further comprise a light emitting diode (LED), for example, an LED that activates when said pressure sensor senses a pressure below a minimum compression threshold.

The sensor may be a bioimpedence sensor, for example, for measurement of limb volume. The bioimpedence sensor may be selected from the group consisting of: an electrochemical electrode, a metallic plunge probe, and a tetrapolar impedance sensor system. The bioimpedence sensor may be a tetrapolar impedance sensor system. The sensor for limb volume may be a sensor for measuring radius of curvature. The sensor for limb volume may comprise one or more circumferential strain gauges, for example, a plurality of strain gauges provided in a plurality of regions on the limb of a subject.

The systems herein may further comprise multiple wireless flexible devices as described herein. For example, the system may comprise four wireless flexible devices, wherein a first device provides an alternating current, a second device is a ground, and additional devices are bioimpedance sensing electrodes capable of measuring differences in voltage. The first device may be placed in closer proximity to a patient's heart than said second device and additional bioimpedance sensing devices are placed between said first device and said second device. The system may further comprise four wireless flexible devices, wherein each device independently has an alternating current signal, a ground, and two bioimpedance sensing electrodes capable of measuring differences in voltage.

The system as described herein may comprise at least three wireless flexible devices. Each of the flexible wireless devices may independently comprise a sensor and a near-field communication system. Each of the wireless devices may be positioned within said functional fabric such that when the covering is worn by a patient the wireless flexible devices contact said patient's gastrocnemius muscle, medial ankle and upper anterolateral thigh.

In order to reduce irritation and provide conformal contact, the flexible wireless devices described herein may have an average thickness of less than or equal to 10 mm, less than or equal to 5 mm, or optionally, less than or equal to 3 mm. The flexible wireless devices, as described herein, may have a lateral area footprint less than or equal to 25,000 mm², footprint less than or equal to 20,000 mm², or optionally, footprint less than or equal to 15,000 mm².

The flexible wireless devices described herein may be encapsulated to protect the device and electronic components from water, perspiration or external environmental hazards such as humidity, heat, cold and the like. The systems described herein may further comprise one or more encapsulating layers. The flexible wireless device or devices may be entirely encapsulated by said one or more encapsulating layers. Each of the encapsulating layers may independently have a thickness less than or equal to 5 mm, less than or equal to 3 mm, or optionally, less than or equal to 2 mm. Each of said encapsulating layers may independently have a geometry without hard edges or concerns that provides for good adherence to the skin and reduced contact stresses, for example, by having a geometry characterized by one or more curved surfaces. Each of the encapsulating layers may independently have a dome shaped geometry, for example, a dome shaped geometry characterized by a fillet radius greater than or equal to 0.2 mm, greater than or equal to 0.25 mm, or optionally, greater than or equal to 0.4 mm.

The encapsulating layers surround the sensor or sensors of the flexible wireless device. For example, the pressure sensor may be encapsulated by a lower encapsulating layer and an upper encapsulating layer forming an encapsulated pressure sensor. The encapsulated pressure sensor may be a piezoelectric sensor, capacitive sensor, a strain gauge sensor, a liquid metal sensor or an iontronic sensor. The upper encapsulating layer may have a thickness at least 1.2 times greater, 1.5 times greater, or optionally, 2 times greater than a thickness of the lower encapsulating layer. One or more encapsulating layers, for example, the lower encapsulating layer may have a geometry characterized by a thickness at least 8 times smaller than at least one lateral dimension, for example, a thickness at least 8 times smaller than a width of said encapsulating layer. The encapsulating layer may be a polymer, for example, polyimide, polydimethylsiloxane (PDMS), polyurethane, polystyrene, polymethyl methacrylate (PMMA) or polycarbonate.

Various forms of wireless communication may be utilized by the flexible wireless device. For example, the wireless communication system may be selected from the group consisting of: a transmitter, a receiver, a transceiver, an antenna, and a near field communication device. The wireless communication system may be one or more near-field coils, for example, near field coils selected from the range of 500 microns to 20 millimeters. Each of the near field coils may independently have an average thickness selected from the range of 1 micron to 5 millimeters. Each of the near field coils may independently have a geometry selected from the group consisting of: an annulus and an elliptical annulus. The wireless communication system may provide Bluetooth wireless communication. The wireless communication system may provide one-way or two-way wireless communication with an external device, for example, a computer, a phone, a smartphone, a tablet or a diagnostic machine.

The systems and electronic devices described herein may rely on a local power source and comprise, for example, a battery or the systems and electronic devices may comprise a wireless energy harvester. The systems and electronic devices may rely on an external power source such as wireless power. The wireless energy harvester may have an area antenna, for example, an area antenna with a length greater than or equal to 50 cm, greater than or equal to 100 cm, or optionally, greater than or equal to 150 cm. The wireless energy harvester may provide a power delivery of greater than or equal to 5×10⁻⁵ mW/cm², 1×10⁻⁴ mW/cm², or optionally, 2×10⁻⁴ mW/cm². The flexible wireless device may or may not further comprise a battery.

The described systems and flexible wireless devices may be useful in telemedicine type applications, in which they provide information regarding treatment or healing to one or more caregivers. The described systems may utilize an external device, for example a computer or smartphone, to record and store sensing data, calculate clinical or real-time metrics and then provide them to the user or caregiver. The described systems and flexible wireless devices may further comprise a processor to provide a real-time metric. The processor may be on-board with the flexible wireless device or be positioned in an external device that is located at a distance from the medical sensor and in wireless communication with the wireless communication system. The system or flexible wireless may continuously monitor and generate a real-time metric. The flexible wireless device may further comprises one or more of a vibratory motor, an electrode, a light emitter, or a thermal actuator.

The systems described herein may comprise a plurality of spatially distributed flexible wireless devices, for example, configured to provide an average output parameter and/or a spatial distribution map of an output parameter. The output parameter and/or spatial distribution map may be time varying. The system may be a multiplexed system configured to provide an output for a plurality of output parameters. The system may be configured to wirelessly communicate output parameters from the system for remote monitoring. The system may further comprise one or more actuators connected to the garment, wherein the actuators are configured to receive an input from a medical person or a feedback input based on one or more sensor outputs from one or more of the sensors.

The provided methods may utilize the various systems, flexible wireless devices, wireless communication systems, configurations, power sources and the like, alone or in combination, as described herein.

In an aspect, provided is a method of measuring an interface pressure of the functional fabric on a region of a body of a subject using any of the systems described herein. In an aspect, provided is a method of treating edema, venous status ulcers, chronic venous insufficiency, deep vein thrombosis, lipodermatosclerosis, lymphedema or any combination of these using any of the systems described herein. The interface pressure may be an instantaneous pressure, an average pressure or a cumulative pressure. Placement of said system in distinct anatomical locations may provide an estimate of a global (e.g., bulk or average) therapeutic pressure over an entire region or skin surface.

In an aspect, provided is a method of assessing fabric failure of the functional fabric using any of the systems described herein. In an aspect, provided is a method of monitoring use of the functional fabric using any of the systems described herein.

In an aspect, provided is a method of providing compression therapy to a subject comprising: i) providing a system as described herein; and ii) administering the system to a limb of the patient. The compression therapy may be for treatment of venous status ulcers, chronic venous insufficiency, deep vein thrombosis, lipodermatosclerosis, lymphedema or a combination thereof.

In an aspect, provided is a method of determining patient adherence comprising: (i) administering a compression therapy system to a subject, the compression therapy system comprising; a) a functional fabric; b) at least one flexible wireless device comprising: a temperature sensor; and a wireless communication system; wherein the functional fabric is provided to a region of the body of the subject; and wherein the at least one flexible wireless device is positioned in mechanical communication with a surface of the functional fabric; (ii) monitoring the temperature sensor to determine the amount of time a patient wears the system thereby determining patient adherence. The compression therapy system may further comprise a pressure sensor. The methods described herein may further comprise a step of determining cumulative pressure provided by combining patient adherence and a time-based output from said pressure sensor

In an aspect, provided is a method of determining compression material failure comprising: (i) administering a compression therapy system to a subject, the compression therapy system comprising; a) a functional fabric; b) at least one flexible wireless device comprising: a pressure sensor; and a wireless communication system; wherein the functional fabric is provided to a region of the body of the subject; wherein the at least one flexible wireless device is positioned in mechanical communication with a surface of the functional fabric; and (ii) monitoring a pressure applied to the subjection by the functional fabric; replacing the system when the pressure falls below a minimum pressure threshold.

In an aspect, a method of determining a limb volume response to an applied pressure in a compression therapy comprising: (i) administering a compression therapy system to a subject, the

compression therapy system comprising; a) a functional fabric; b) at least one flexible wireless device comprising: a pressure sensor; a wireless communication system; and a sensor for measurement of limb volume; wherein the functional fabric is provided to a limb of the subject; and wherein the at least one flexible wireless device is positioned in mechanical communication with a surface of the functional fabric; (ii) measuring a pressure applied to the subject; and measuring a limb volume response to the applied pressure.

In an aspect, provided is a method of measuring a real-time metric of a patient comprising the steps of: a) contacting a patient with a therapeutic garment and at least one flexible wireless device; b) detecting a signal generated by said wireless device; c) analyzing said signal to generate a real-time metric; and d) providing said real time metric to the user or a third party. The real-time metric may be generated from limb volume, pressure, temperature, patient adherence, sweat analysis or any combination thereof.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1A provides an example schematic of a capacitive pressure sensor.

FIG. 1B shows an example of a flexible wireless device applied to the skin.

FIG. 1C and FIG. 1D show compression stockings which are examples of therapeutic garments.

FIG. 1E and FIG. 1F show examples of flexible wireless sensing devices.

FIG. 1G and FIG. 1H show exemplary data output from a flexible wireless device detecting pressure.

FIGS. 2A-2B provides examples of tetrapolar bioimpedance measuring devices. FIG. 2A is a tetrapolar device on a single electronic device. FIG. 2B is a tetrapolar system provided on four separate electronic devices.

FIG. 3A illustrates Hammocking error which may occur when the Young's modulus of the sensor is substantially different than the modulus of the adjacent skin.

FIG. 3B provides a schematic of various flexible wireless devices described herein.

FIG. 3C shows contact stress on the skin of a calf portion of a person's leg from the devices described in FIG. 3B illustrating the benefit of the larger PDMS base.

FIG. 3D shows sensitivity of the embodiments described in FIG. 3B.

FIG. 3E illustrates experimental set up for testing the various devices illustrated in FIG. 3B.

FIG. 3F provides experimental data regarding applied pressure and measured conductance of the various devices of FIG. 3B.

FIGS. 4A-4B provides an illustration of determining leg volume using a strain gauge sensor. FIG. 4A illustrates that the pressure exerted by the stocking (p₂) is assumed equivalent to the opposite pressure exerted by the leg (p₁). FIG. 4B illustrates calculating the radius of limb due to deformation of the device.

FIG. 5 provides an example of the compression garment standard of care.

FIG. 6 provides an example pressure sensor and its specifications used in the research developing the devices and methods described herein.

FIG. 7 illustrates two additional exemplary devices.

FIG. 8 provides a pressure sensor as described in FIG. 6 configured to communicate with a mobile device.

FIGS. 9A-9B shows the deformation of interface region due to intrusive rigid sensor. The “Hammocking” effect tension devices such as bandages and combat application tourniquets (FIG. 9A) and pneumatic tourniquet type devices (FIG. 9B).

FIG. 10 provides an illustration of the physical interaction between the sensor and limb and various variables as used herein.

FIGS. 11A-11C illustrates that the pressure underneath the compression sock is proportional to the strain of the sock and inversely proportional to the radius of curvature. FIG. 11A pressure is uniform around the leg. FIG. 11B pressure is higher at p₁ and lower at p₂. FIG. 11C pressure is negligible at p₂.

FIGS. 12A-12B provides the physical interaction between the sensor and limb and various variables as used herein. FIG. 12A shows a compression stocking without a sensor. FIG. 12B is a compression stocking with a sensor.

FIGS. 13A-13B illustrates equilibrium forces for a compression stocking. FIG. 13A corresponds to a compression stocking without a sensor as provided in FIG. 12A. FIG. 13B corresponds to a compression stocking with a sensor as provided in FIG. 12B.

FIG. 14 provides a top and cross-sectional view of an example wireless device configuration as described herein.

FIGS. 15A-15C provides additional embodiments which enhance sensor sensitivity. FIG. 15A includes a series of pillars in the pocket. FIG. 15B includes ionic liquid within the pocket. FIG. 15C includes a bump on the top of the pocket.

FIGS. 16A-16C provides additional detail of the designs described in FIGS. 15A-15C which provide maximum strain by pressure and minimally affect the transverse strain of the compression stocking or skin. FIG. 16A pillars. FIG. 16B ionic liquid. FIG. 16C bump on top of the pocket.

FIG. 17 provides additional features which may increase the sensitivity or effectiveness of the wireless devices described herein. FIG. 17A Pneumatic structure. FIG. 17B Carbon black strain gage. FIG. 17C compression stockings sensor.

FIGS. 18A-18C provides examples of currently available commercial sensors. FIG. 18A capacitive sensor. FIG. 18B piezoelectric sensor. FIG. 18C fabric based sensor.

FIG. 19 provides an example of an encapsulated sensor.

FIG. 20 provides an example circuit diagram of the wireless device/sensor.

FIG. 21 describes the optimization of signal detection.

FIGS. 22A-22B provides example wireless devices referred to as epidermal interface pressure sensors. FIG. 22A EIPS—Type A. FIG. 22B EIPS—Type B.

FIG. 23 describes the components of an EIPS and provides examples of the two types shown in FIGS. 22A-22B.

FIG. 24 describes a configuration for calibrating an EIPS.

FIG. 25 shows the results of the calibration testing described in FIG. 24 using a high sensitivity setting (340 k Ω) and the calibration curve established from the results.

FIG. 26 shows the results of the calibration testing described in FIG. 24 using a broad range setting (100 k Ω) and the calibration curve established from the results.

FIG. 27 describes another configuration for calibrating an EIPS.

FIG. 28 shows the results of the calibration described in FIG. 27. The results are both repeatable and highly linear.

FIG. 29 illustrates the effect of encapsulation of the wireless device on repeatability.

FIG. 30 illustrates the effect of encapsulation of the wireless device on repeatability.

FIG. 31 provides an example of a non-elastic compression stocking test and pressure results provided by a device as described herein.

FIG. 32 provides an example of an elastic compression stocking test.

FIG. 33 provides the results of the elastic compression stocking test provide in FIG. 32.

FIG. 34 provides an example of a test comparing different systems of measurement with the wireless devices described herein.

FIG. 35 provides the results of the output measurement comparison described in FIG. 34.

FIG. 36 provides an example of a compression stocking kit with a plurality of wireless devices and important positions within the kit.

FIG. 37 provides two examples of near field communication (NFC) epidermal electronics which provide wireless, battery-free operation.

FIG. 38 illustrates the advantages of near field communication electronics which is a short-range wireless communication system found in many smartphones and other comparable devices, often used in medicine, the military, transportation and banking. All NFC devices have a unique ID for authentication.

FIG. 39 illustrates communication between a compression stocking and a computer using NFC.

FIG. 40 illustrates that a unique ID scan can be performed on multiple NFC enabled devices after their removal from the patient (up to 60 unique devices).

FIG. 41 provides examples of the many commercial and custom NFC readers available.

FIG. 42 provides examples of thin, flexible form factors.

FIG. 43 provides examples of wireless devices useful in compression garments that are capable of digital volumetric sweat analysis and temperature sensing. NFC designs can operate under extreme conditions.

FIG. 44 provides an example of a manufacturing and development process for generating NFC devices as described herein.

FIG. 45 provides an example of a manufacturing and development process for generating NFC devices as described herein.

FIG. 46 provides an example useful manufacturing tools for generating NFC devices as described herein.

FIG. 47 provides additional examples of manufacturing processes which may be useful in creating the wireless devices as described herein.

FIG. 48 illustrates that sensors may be formed with aesthetic designs via rapid prototyping.

FIG. 49 provides an example of a wireless device as described herein that incorporates light emitting diodes and bioimpedance.

FIG. 50 provides an example of a wireless device as described herein with an integrated temperature sensor.

FIG. 51 provides examples of other garments with integrated, flexible, wireless devices such as bandages, wound dressings (e.g. pressure ulcer wound healing products), abdominal binders and athletic gear (e.g. compressive pants, shirts and sleeves).

FIG. 52 provides a therapeutic garment incorporating one or more wireless devices.

FIG. 53 provides a top and cross-sectional view of an example flexible wireless device.

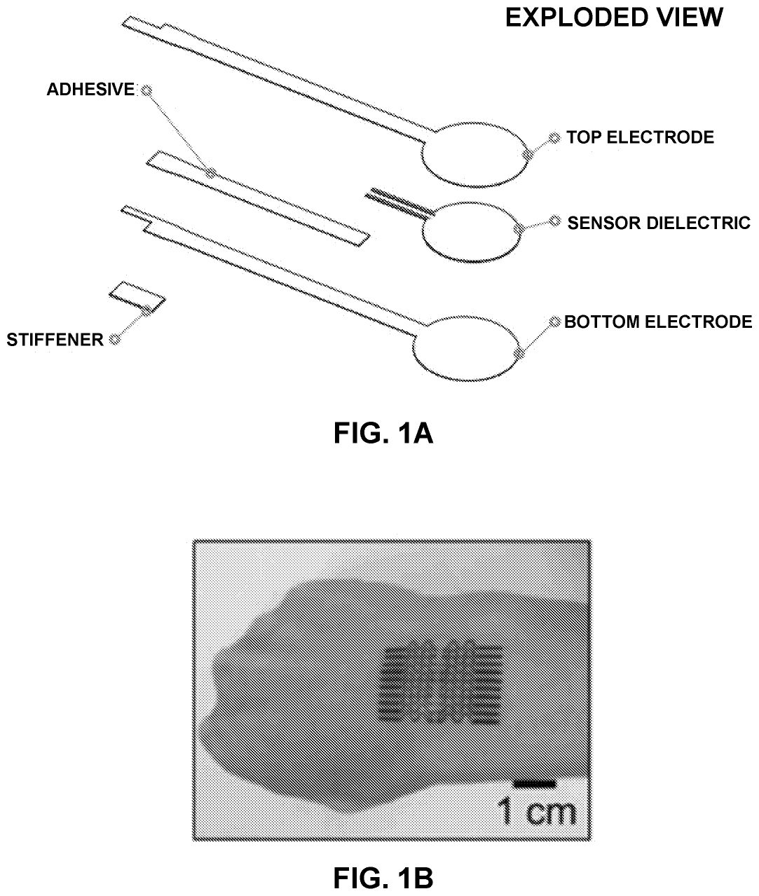

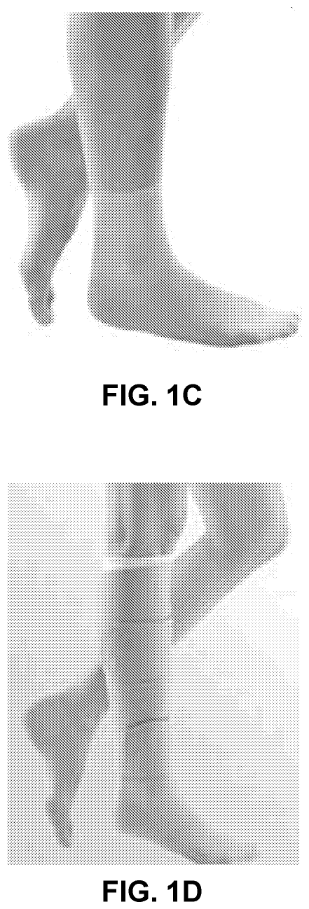

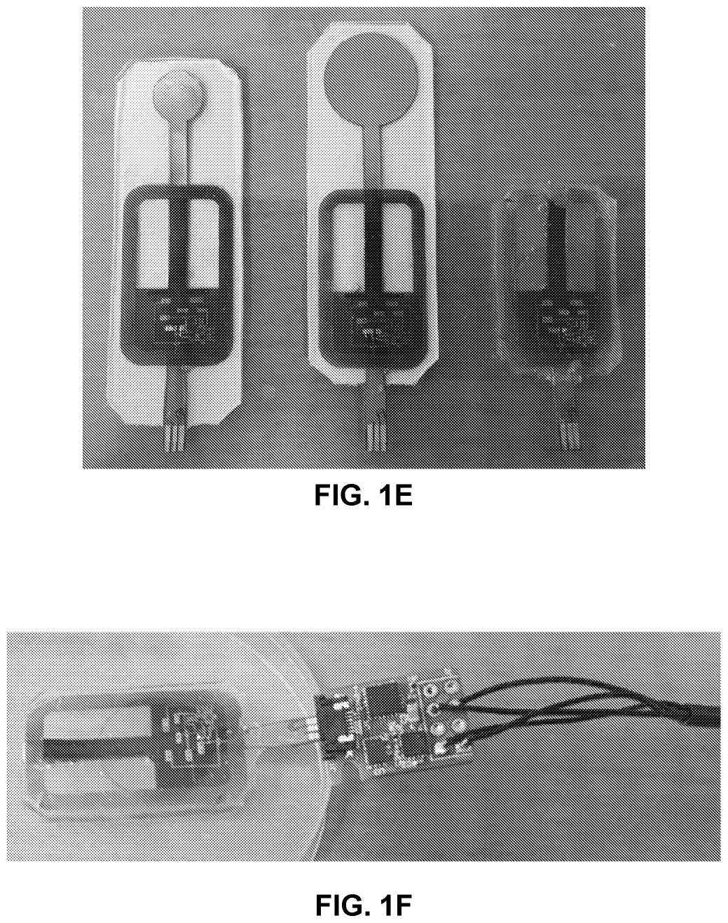

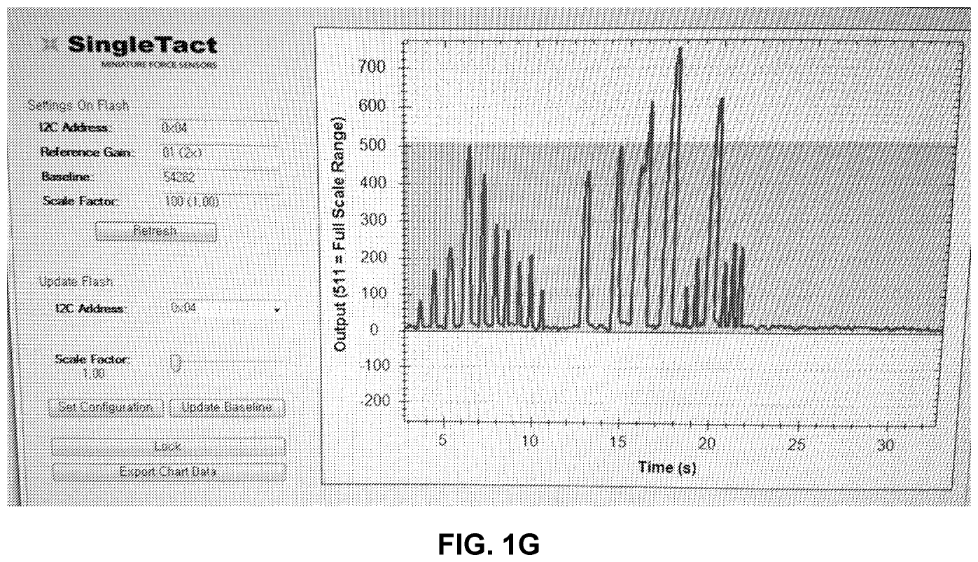

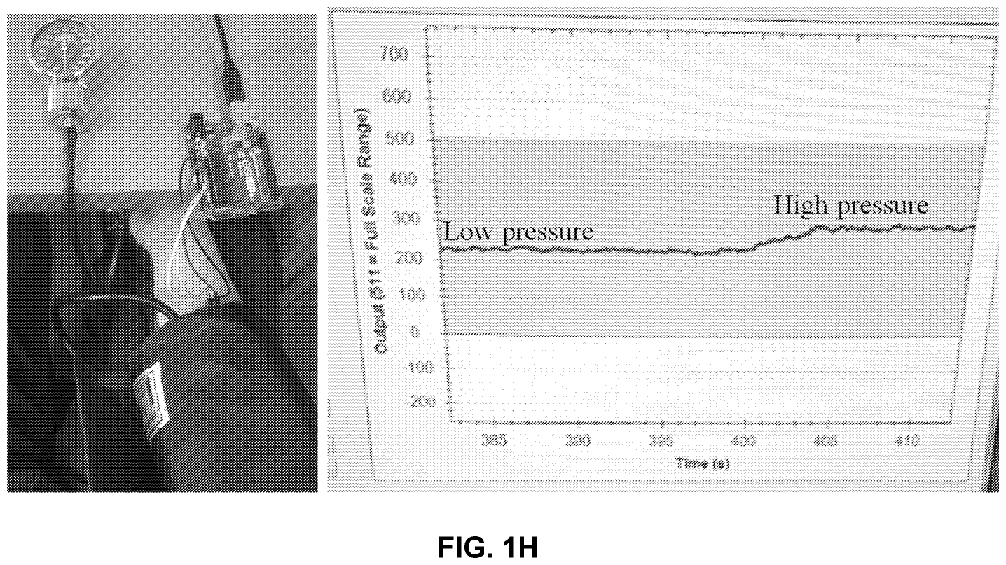

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Functional fabric” refers to a substrate having an inner facing and outer facing surface, with the inner facing surface directed to a user's skin and the outer facing surface away from the skin, with a material thickness separating the surfaces. The term is used broadly herein, reflecting that the devices and methods described herein have a broad range of applications. For example, the functional fabric may be a textile or garment. The terms textile and garment may be used interchangeably with both a “therapeutic garment” and a “diagnostic garment.” Functional fabric may be, for example, a medical wrap, bandage, medical garment, a compression garment, an inelastic functional fabric, an elastic functional fabric, a woven functional fabric, a non-woven functional fabric or a knit functional fabric. Garment may be a sock, stocking, sleeve, glove, shirt, tights, skull cap, or other type of clothing. Garment may also refer to wraps, bandages, wound dressings, braces, hard casts, soft casts, splints, pneumatic compression devices and other medical devices. A functional fabric as described herein may be a therapeutic compression stocking. The functional fabric may be moistened, heated or cooled, including a precise manner and/or feedback-type loop, with one or more sensors providing an input used to drive one or more actuators to provide desired functionality. Functional fabric may also refer to performance or smart garments, for example, performance athletic wear with additional smart properties such as cooling, heating, aiding recovery or reducing muscle soreness (e.g. reducing lactic acid), preventing injury, sweat wicking, adding moisture, compression, antibacterial properties, polymer coatings and the like. The functional fabric may be capable of a therapeutic functionality, such as compression, would healing or reduction of muscle soreness. The functional fabric may have a diagnostic and/or monitoring function, such a monitoring or adjusting a physical or physiological parameter such as temperature, pressure, blood oxygen, UV exposure, limb volume and the like. Accordingly, the term “functional” in a functional fabric refers to a fabric having one or more sensors to provide useful and actionable information, with that information specific for an application of interest. For example, confirmation of pressure, length of time worn, an environmental parameter such as heat, light intensity, moisture, a skin-specific property such as oxygenation, blood flow, skin discoloration or redness.

“Pressure sensor” refers to a device capable of generating a signal corresponding to pressure generated between the compression covering and the tissue or skin of a wearer of the compression covering. Pressure sensors may include capacitive pressure sensor or peizoresistant sensors. In embodiments, pressure sensors have a thickness of less than or equal to 10 mm, less than or equal to 5 mm, less than or equal to 2.5 mm, or optionally, less than or equal to 1 mm. Pressure sensors, in some embodiments, may further measure circumferential leg pressure, for example, to estimate leg volume.

“Communication system” refers to components or systems which allow the device to send a signal to monitoring device, for example, a personal computer or smartphone. In embodiments, communication systems are selected from the group consisting of a transmitter, a receiver, a transceiver, an antenna, and a near field communication device. Communication system may include devices capable of Bluetooth communication.

“Barrier layer” and/or “encapsulating layer” refer to one or more layers configured to prevent moisture from reaching the electronic components, for example, the pressure sensor, the limb volume sensor and/or the near field communications chip. Encapsulating layers may surround a device or device component whereas barrier layers may protect one surface or side of a component. In embodiments, a substrate and a barrier layer may combine to form an encapsulating layer. In embodiments, encapsulating layers have a thickness less than or equal to 5 mm, less than or equal to 2.5 mm, or optionally, less than or equal to 1 mm.

“Without substantial inflammation, irritation or discomfort” refers to the capability of the present sensors to contact a skin or tissue surface without causing patient discomfort or skin irritation, for example, wounds, rash, scaling, inflammation and/or immune response. In some embodiments, for example, the sensor may have properties and dimensions such that a patent wearing a compression covering cannot feel the sensor, or the sensor impact is negligible. The term refers to at least there being no observable adverse impact to the tissue surface by the naked eye.

“Real-time metric” is used broadly herein to refer to any output that is useful in medical well-being. It may refer to or be derived from temperature, pressure, limb volume, skin contact, patient adherence, sweat analysis or any other patient parameter as described herein. Real-time metrics may be derived values such as temperature-pressure maps generated by a network of wireless devices positioned in contact with a patient under a therapeutic garment. Real-time metric may refer to a clinical metric, which provides a caregiver with additional insight into the health or healing of a patient. A real-time metric may also be referred herein generally as a “parameter” that is measured by a sensor, including a parameter relevant for a therapeutic assessment. Examples include, but are not limited to, one or more of: pressure, limb volume, tissue coloration (e.g., redness), temperature, force, stress, blood flow, oxygenation, position or location, strain, motion, elapsed time worn, total number of times garment put on and taken off. Any of the systems and methods may be described as multiplexed, including having a plurality of devices for measuring a plurality of parameters, including real-time metrics.

“Conformal integration” refers to the ability of the present systems to be provided to a tissue in a manner that the device spatially conforms at an interface between the system and the tissue or at the interface with an intermediate structure provided between the system and tissue surface. Conformal integration may be via direct or indirect contact with a tissue surface.

“Spatially Distributed” refers to positioning multiple flexible wireless devices such that they take measurements via sensors different positions on the skin, on a single limb or body part, or on multiple body parts. Spatial distribution may be designed to provide additional information to the user or caregiver my targeting specific regions, muscles, body parts or to provide error checking by comparing and contrasting each of the measurements. Spatially distributed may refer to distribution within a therapeutic garment, a patient or part of a patient, or a combination thereof.

“Output parameter” refers to a signal output of one or more physical properties or conditions measured by the flexible wireless devices as described herein. Output parameter may refer to temperature, pressure, limb volume, bioimpedence, patient adherence or any other property or condition as described herein. Output parameters may be calculated values or metrics, for example real-time metrics, using one or more additional output parameters or measurements to derive or combine measurements into more complex or informative parameters.

“Spatial distribution map” refers to combining a plurality of output parameters captured by a plurality of spatially distributed flexible wireless devices to generate a two or three dimensional map the output parameters.

“Actuator” refers to a component of a flexible wireless device that is capable of interacting with the user of the device, the therapeutic garment or the surrounding environment. For example, an actuator may be a vibratory motor, an electrode, a light emitter, a screen or a thermal actuator. Actuators may engage or interact based on an external signal, for example, from a user or caregiver. Actuators may be engaged via an electronic device (laptop, smartphone, etc.) in wireless communication with the flexible wireless device. Actuators may be useful in telemedicine style treatments for allowing a caregiver to remotely engage or disengage actuators as part of treatment or therapy.

“Feedback input” refers to a means of engaging or disengaging a component of the flexible wireless device (e.g. a signal or actuator) that is generated by a sensor or processor of the wireless device. For example, a pressure sensor may provide a feedback input to a LED when the pressure falls below therapeutic levels to alert the user or medical professional that the therapeutic garment should be replaced or repositioned.

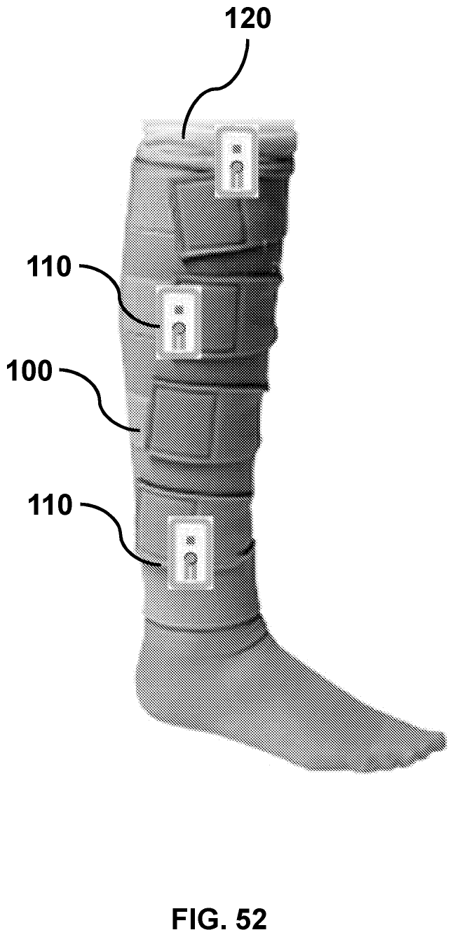

FIG. 52 provides a therapeutic garment incorporating one or more wireless devices. A therapeutic garment 100 contains one or more flexible wireless devices 110 in mechanical communication with the therapeutic garment 100 such that they can establish conformal contact with a patient's skin or limb 120.

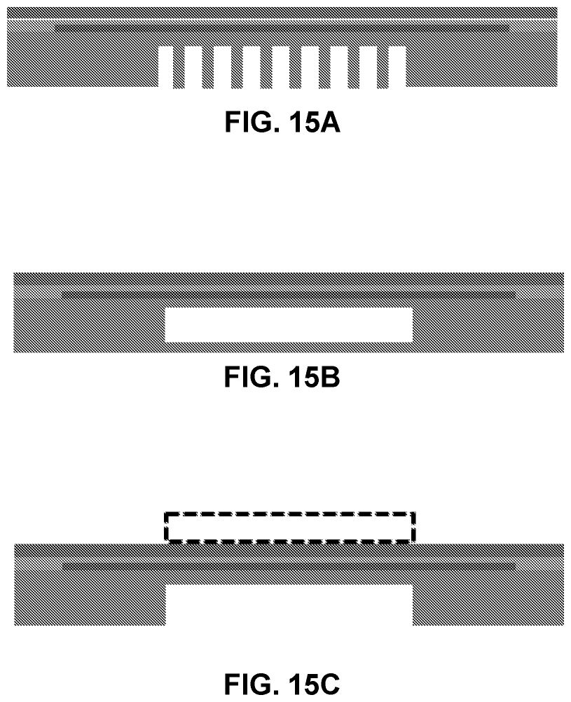

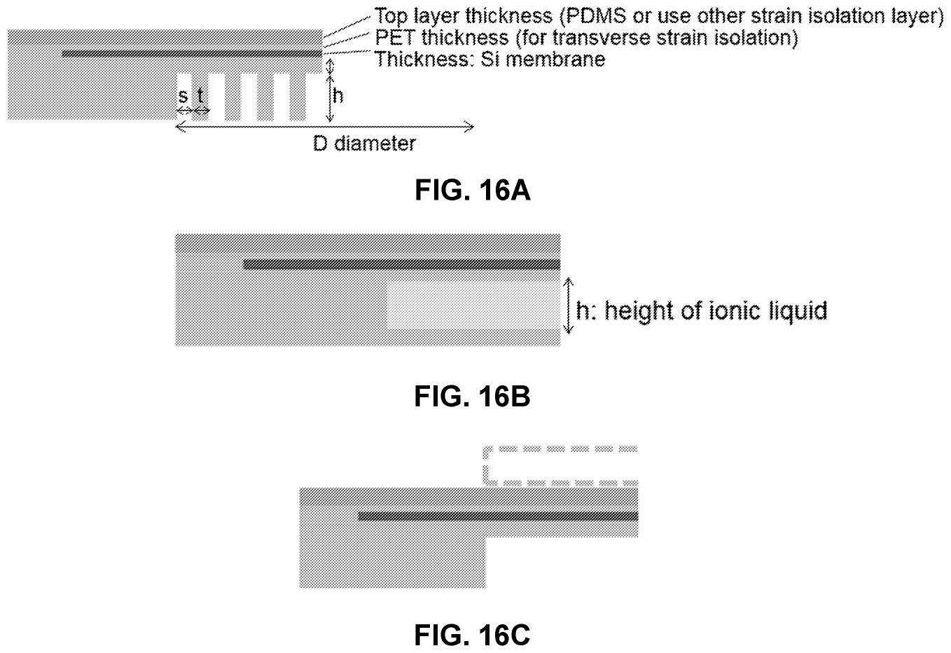

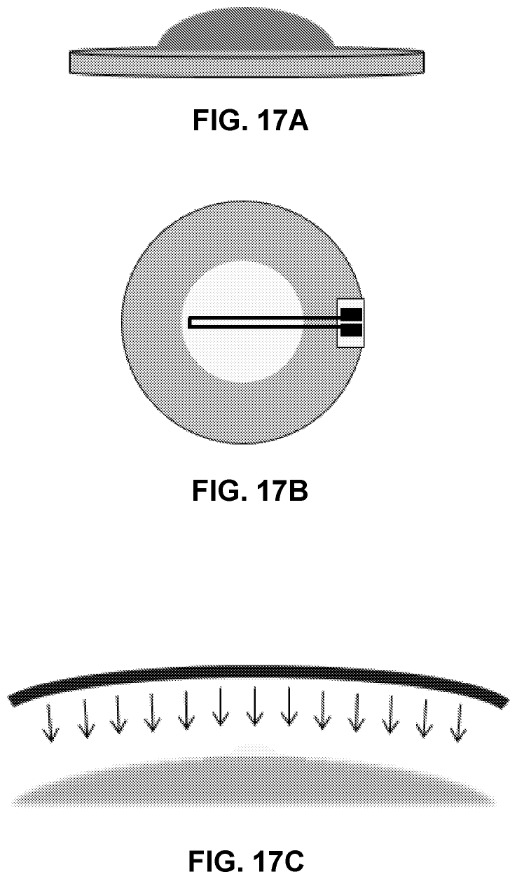

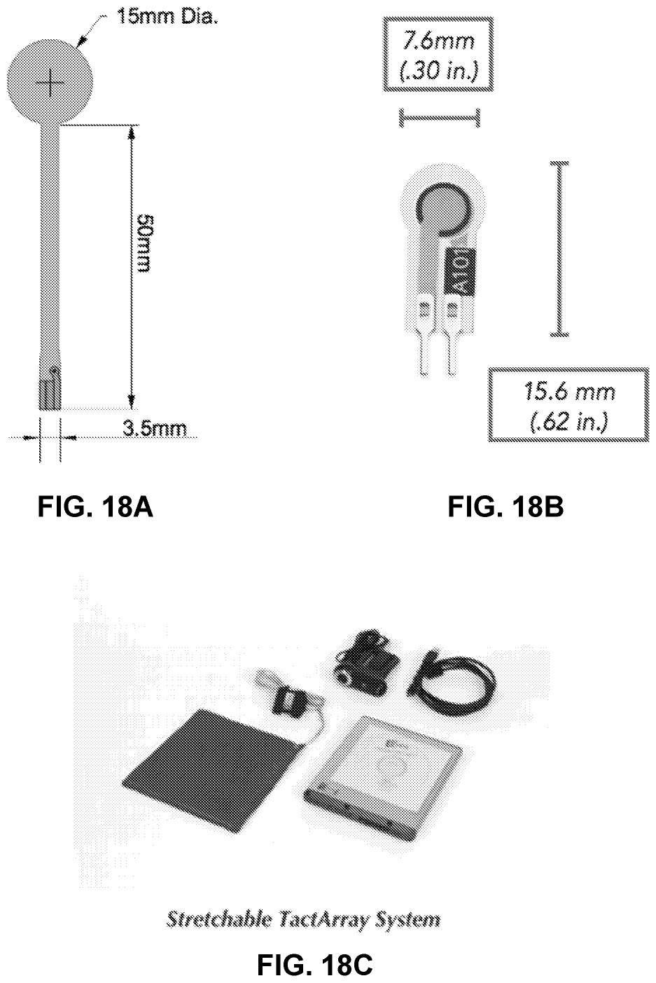

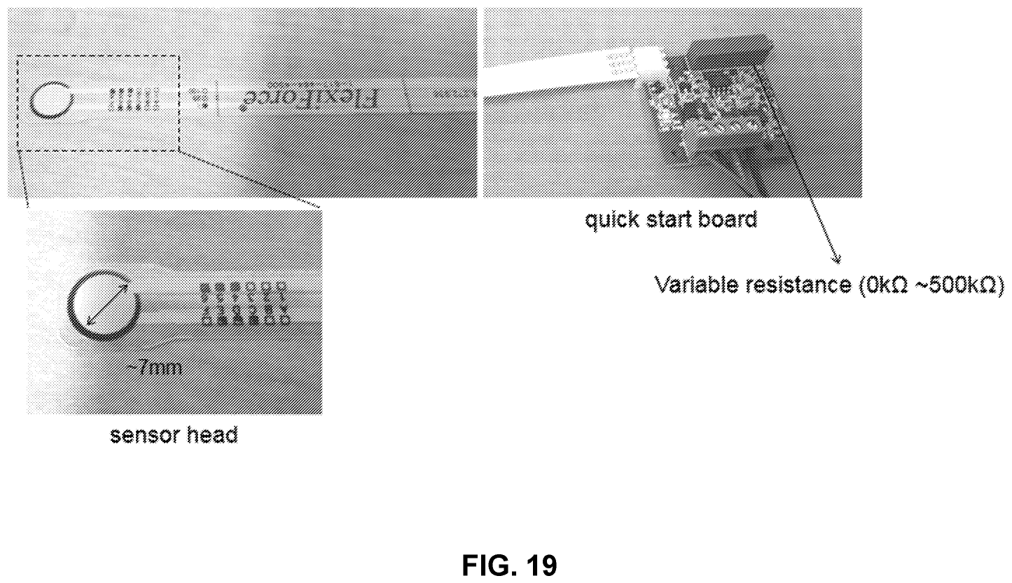

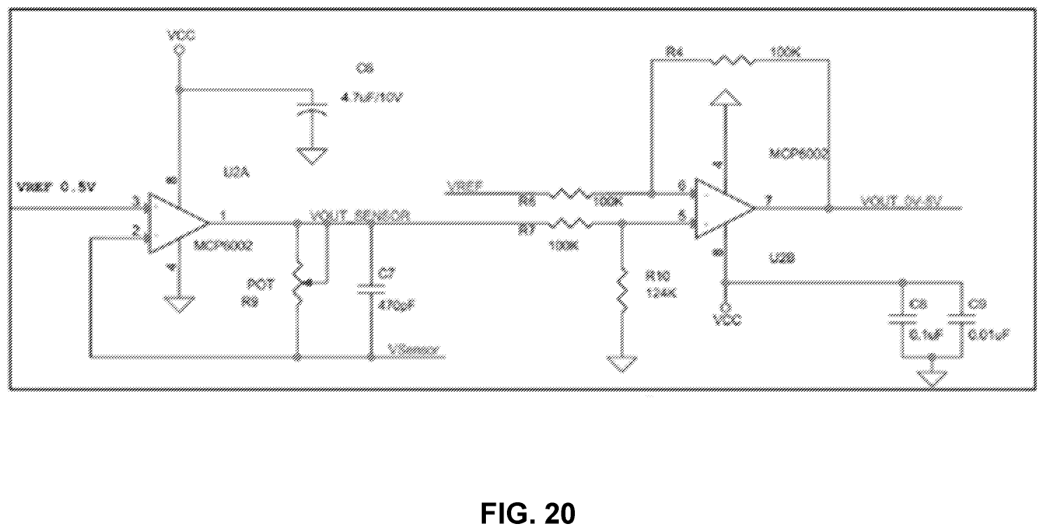

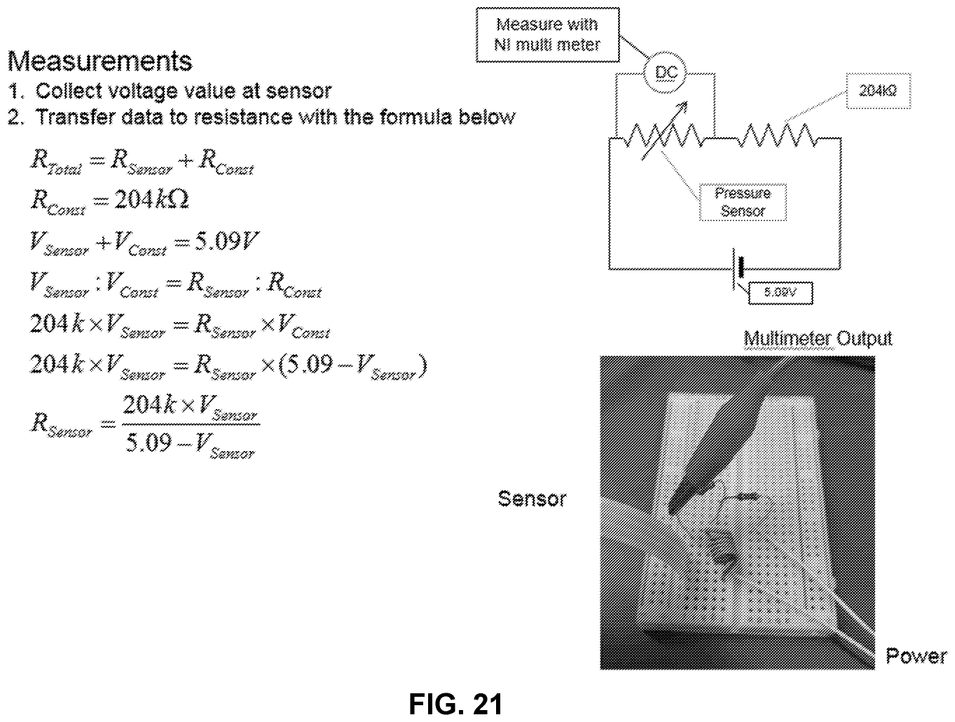

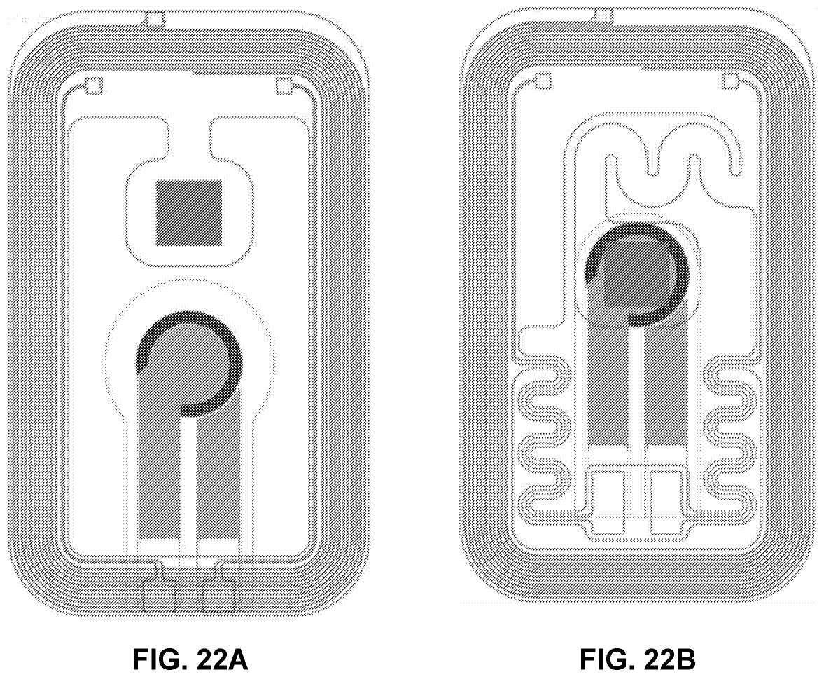

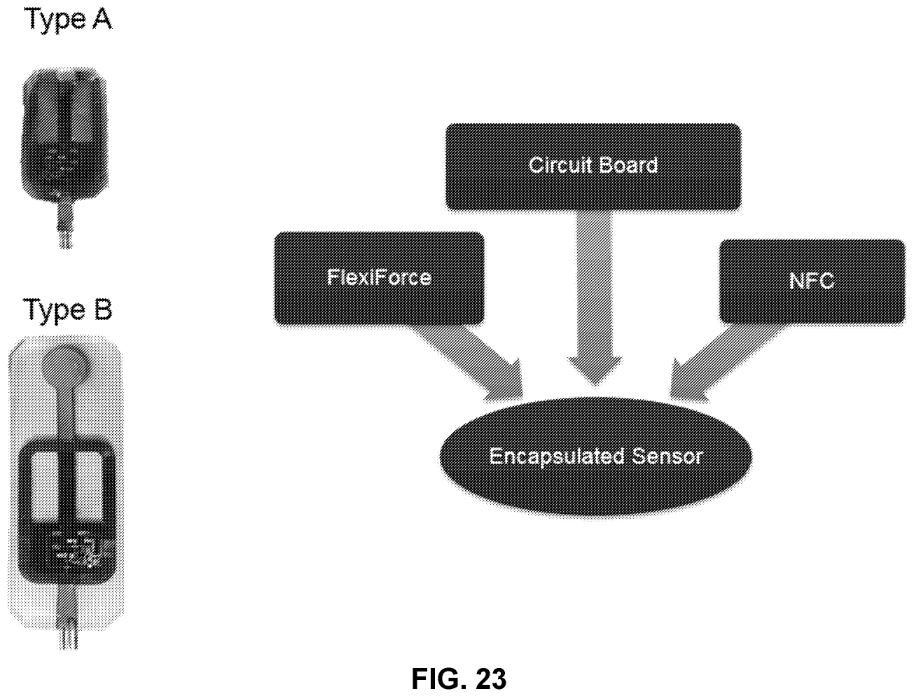

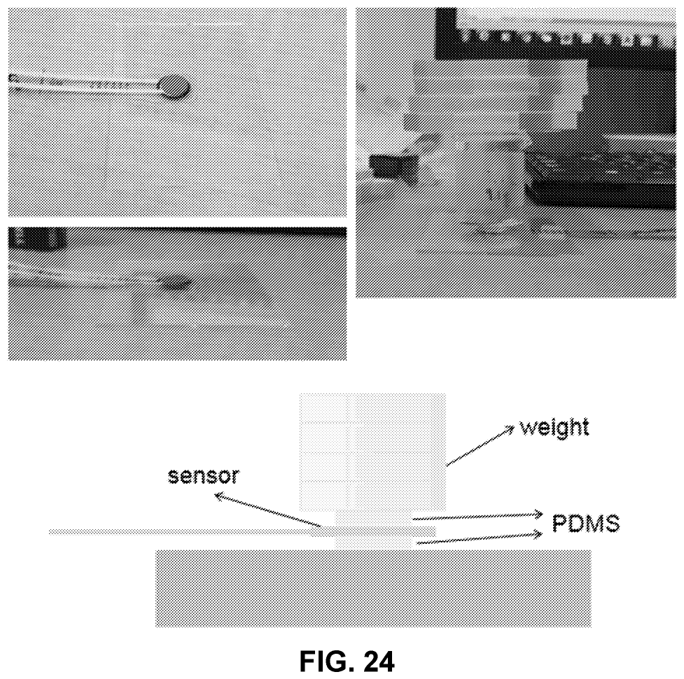

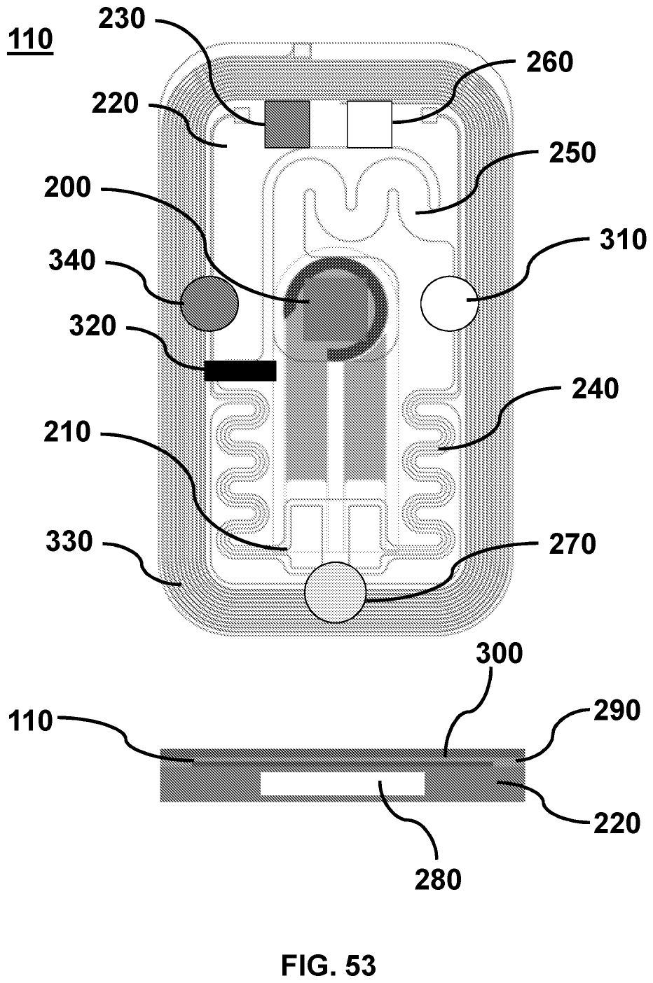

FIG. 53 provides a top and cross-sectional view of an example flexible wireless device. The flexible wireless device 110 includes a pressure sensor 200 and a wireless communication device 210 positioned on a substrate 220. The flexible wireless device 110 may include additional sensors, for example, a sensor for the measurement of limb volume 230, a wireless power source 240, a microfluidic sensor 250, a bioimpedence sensor 320 and/or a temperature sensor 260. The flexible wireless device 110 may also include a battery 270 to provide local power. The substrate 220 may have a void space 280 to incorporate additional features such as an ionic liquid or pillars (as shown in FIGS. 15A-15B). A superstrate 290 may be positioned proximate to the flexible wireless device 110 to protect the portion of the flexible wireless device 110 not protected by the substrate 220. The flexible wireless device may further include one or more encapsulating layers 300. One or more electrical interconnects 330 (e.g. flexible electrical interconnects) may operable connect the various components of the flexible wireless device 110 to form electrical circuits.

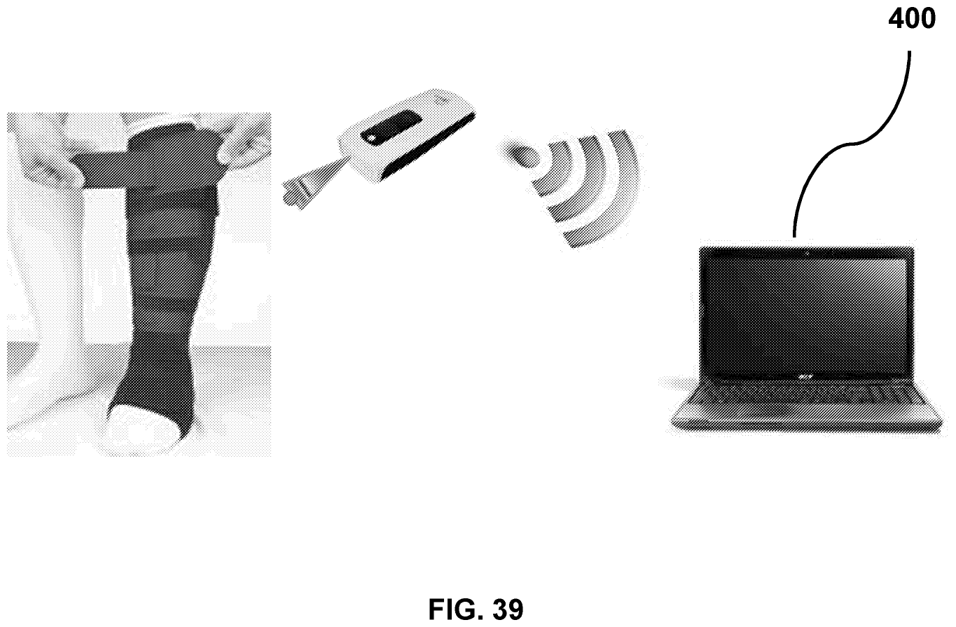

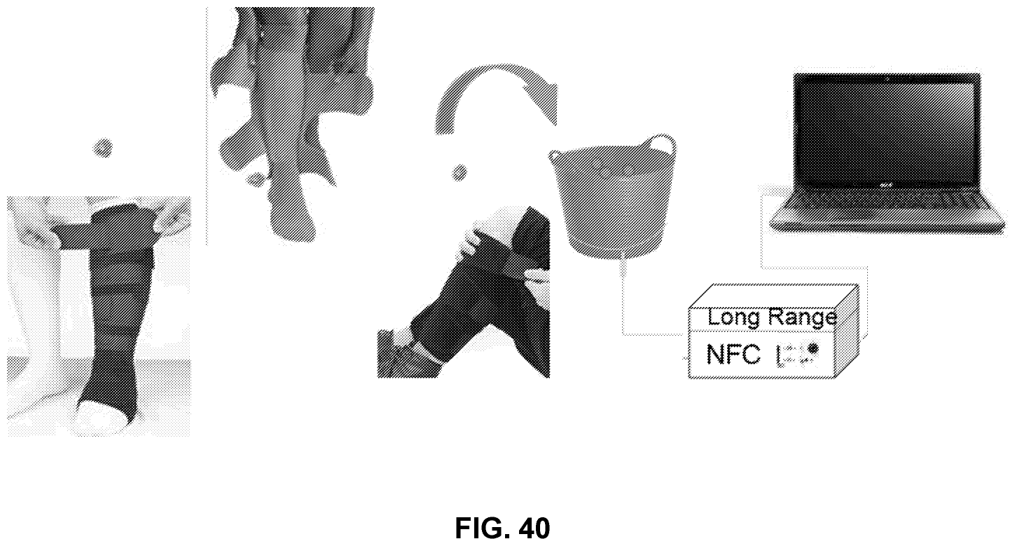

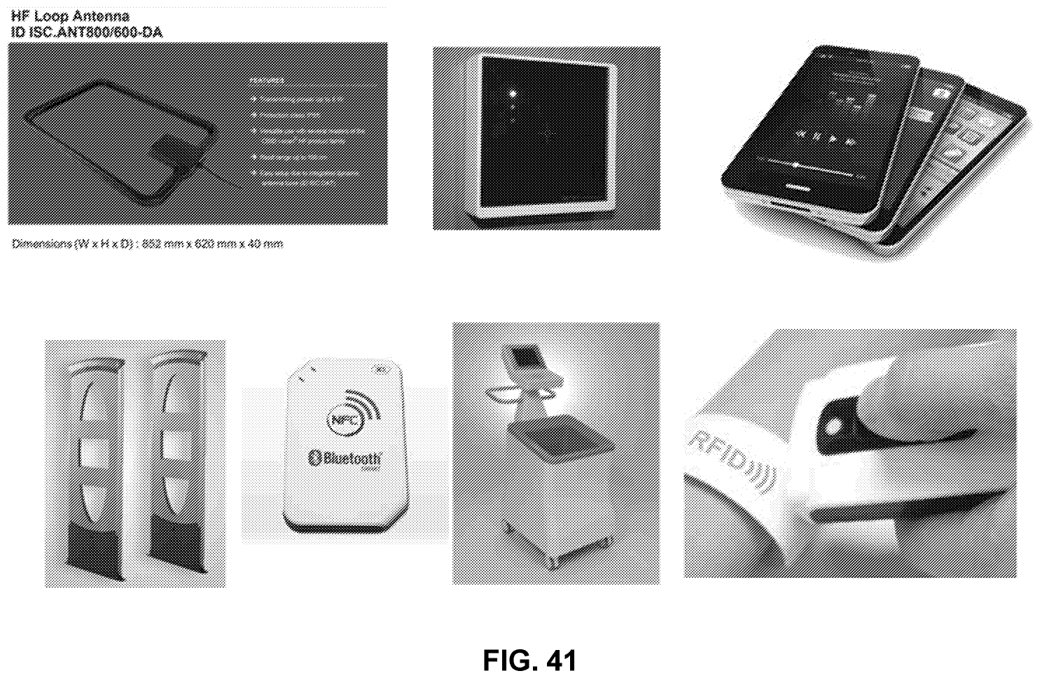

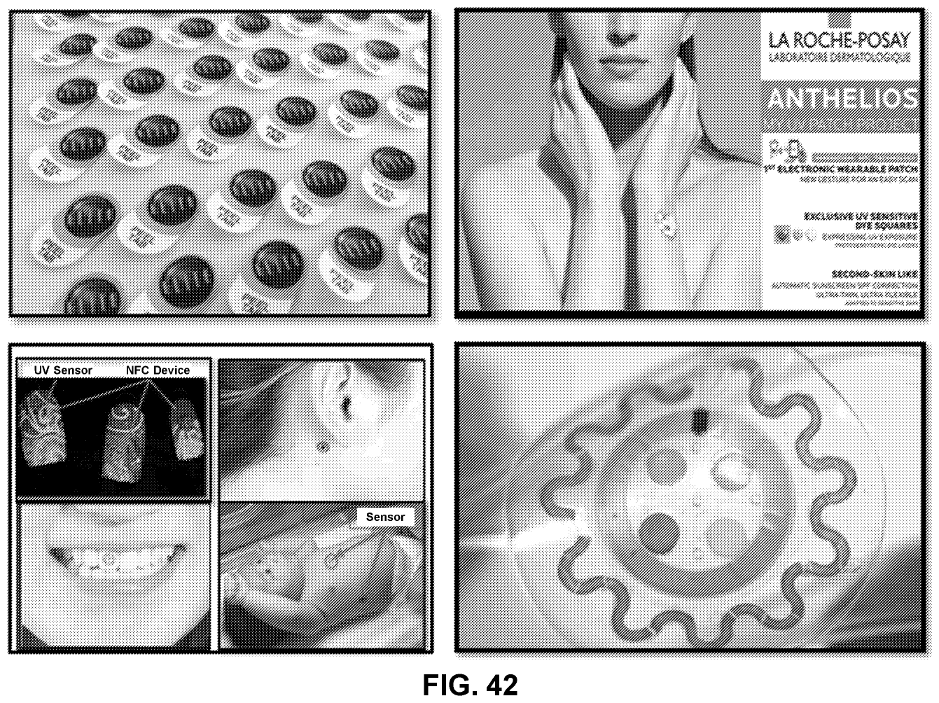

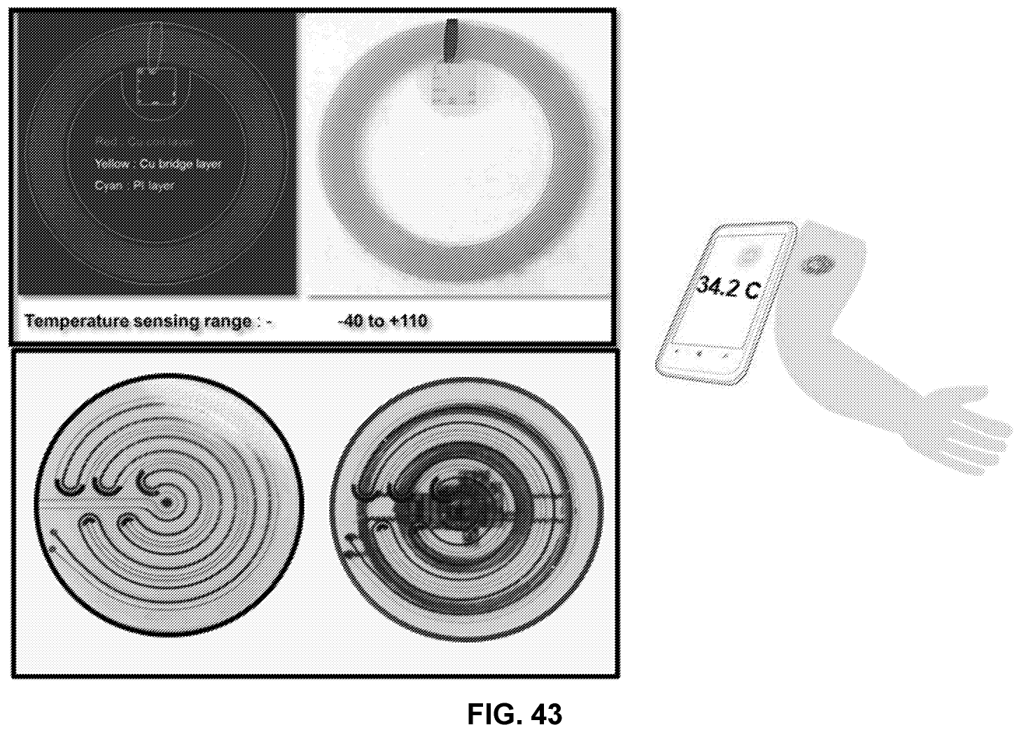

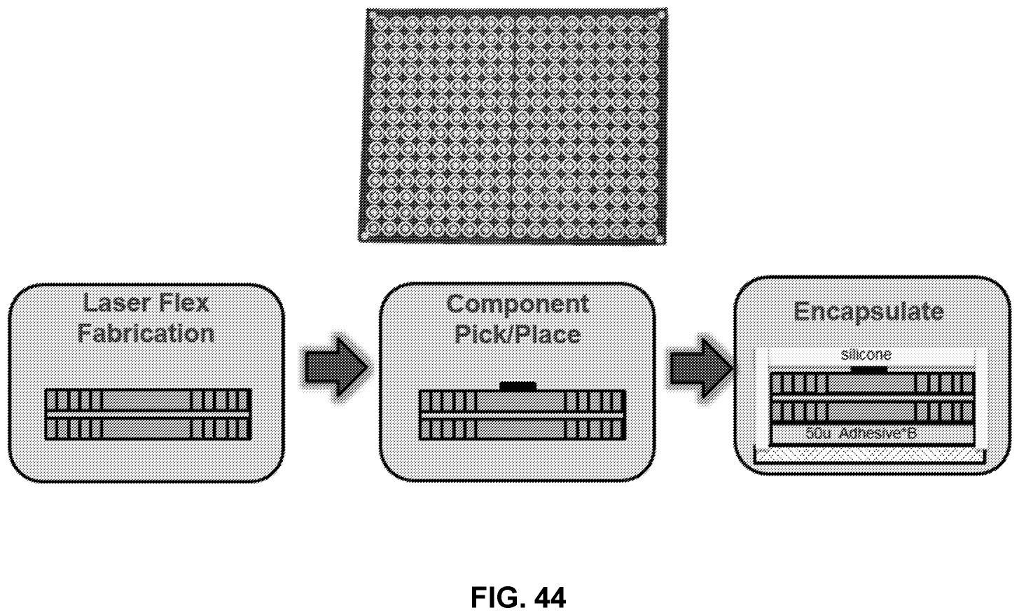

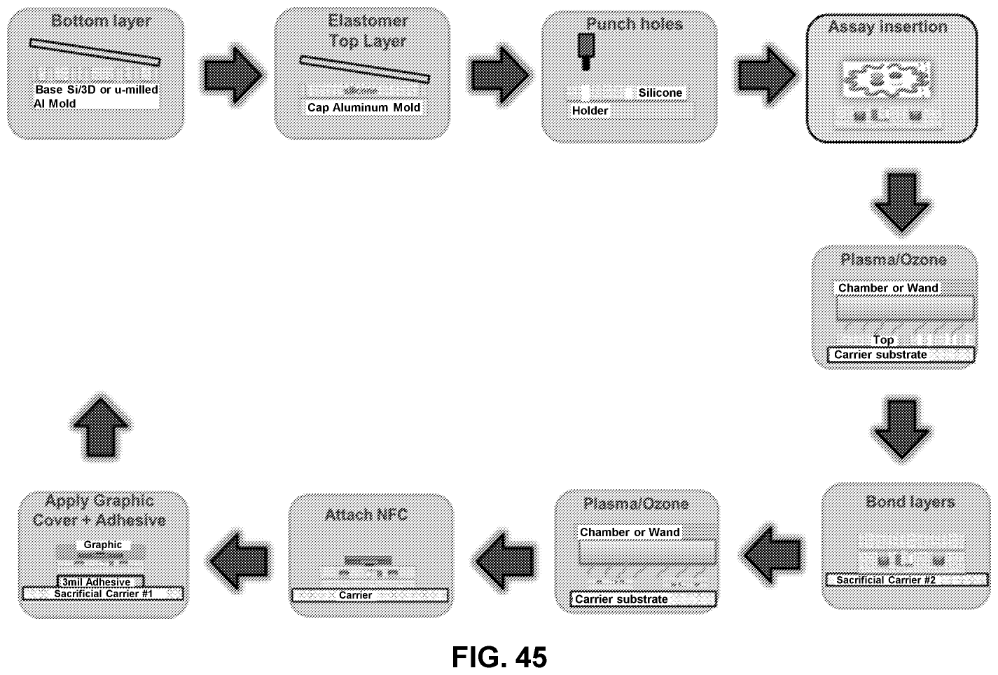

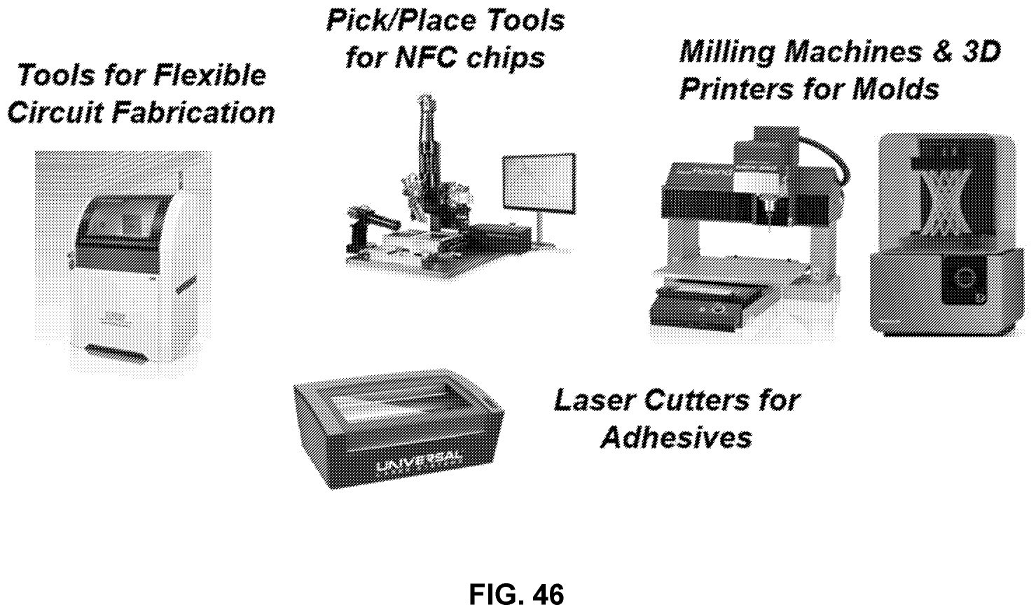

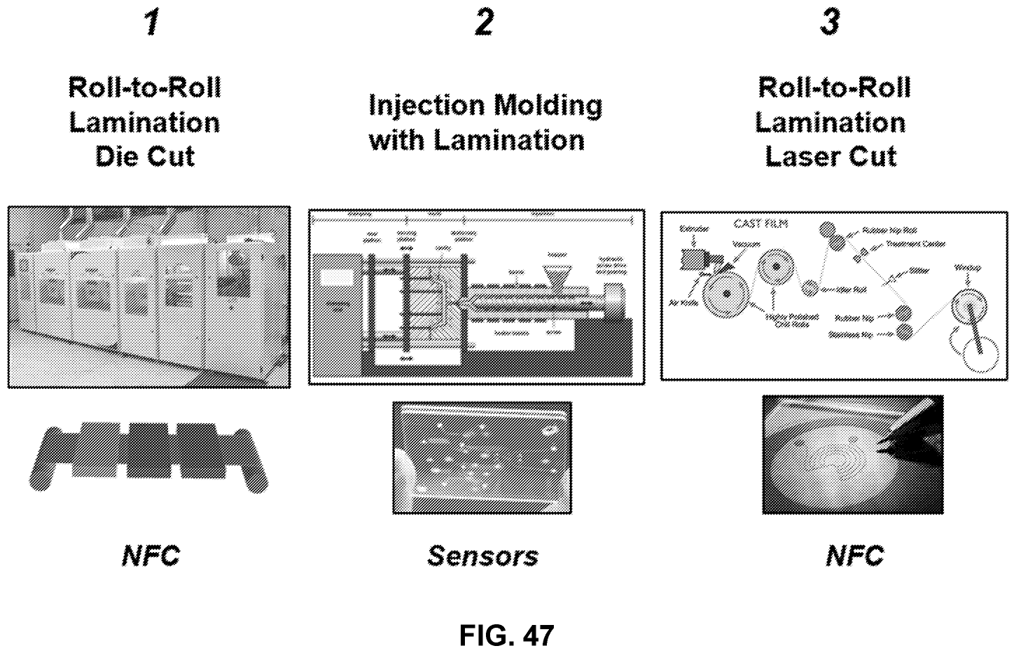

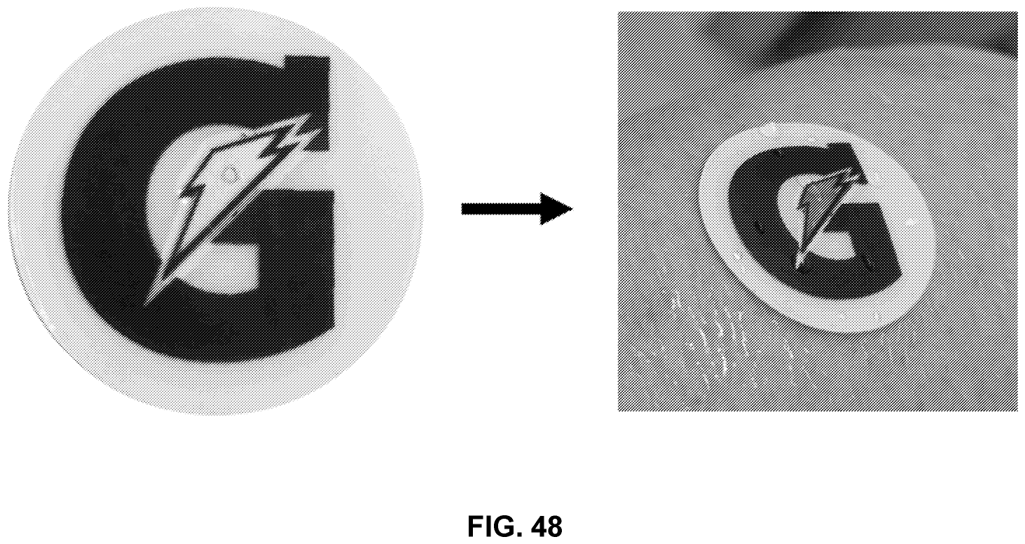

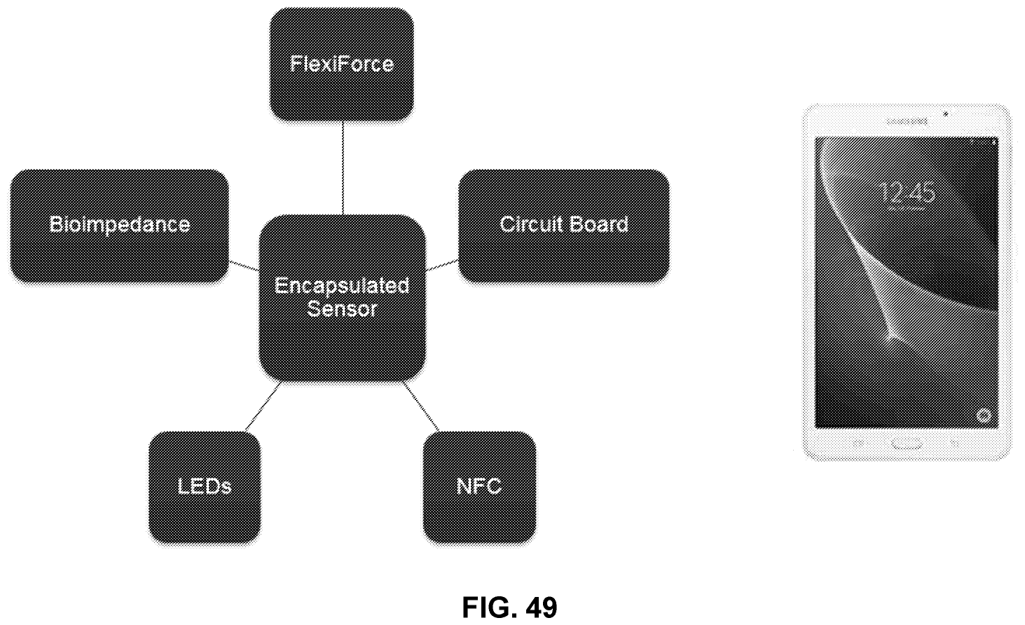

In order to generate clinical metrics, real-time metrics or spatial distribution maps the flexible wireless device 110 may include an on-board processor 310 or use wireless communication to provide a signal to an external processor, for example, a laptop 400 (as seen in FIG. 39). The flexible wireless device 110 may also include a signaling device 340 (e.g. an LED) to signal to a user or caregiver clinical information detected by the flexible wireless device.

### Example 1: Treatment of Venous Stasis Ulcers by Compression Therapy

More than 500,000 Americans suffer from venous stasis ulcers (VSUs), a condition driven by lower extremity venous hypertension, which costs the U.S. healthcare system more than $1 billion annually. The cornerstone of treatment involves the use of medical compression stockings that deliver therapeutic pressure to the lower extremities. In this example, the described therapeutic garment is a compression stocking. Compression stockings aid in venous return and promote wound healing. Recent meta-analyses and consensus statements from leading venous medicine societies have shown that compression stockings both speed healing for VSUs and prevent their recurrence. Furthermore, higher therapeutic pressures can be more effective in promoting VSU healing.

Garment manufacturers grade the strength of their products (e.g. 10-20 mmHg, 20-30 mmHg, 30-40 mmHg) based on tensile strength testing in vitro. Currently, there are no commercially available medical compression stockings that provide in vivo feedback of therapeutic pressure. Interface pressure measurements have been largely limited to research settings given their cost, bulkiness and unsuitability for continuous wear. Even though a garment may be rated the same strength, the actual therapeutic pressure varies with the patient's skin collapsibility, volume changes with body position, garment fit and patient adherence. Thus, there is a clinical need for a smart compression stocking device that is able to provide real-time feedback of interface pressure to ensure adequate therapeutic pressure and patient adherence.

Patients with VSUs have extremely delicate skin. Rigid and sharp electronic devices may pose risks to the patent, including infection. Large, bulky systems will also likely negatively impact patient adherence and comfort. The Rogers group has pioneered the development of flexible electronic systems that are mechanically invisible, highly stretchable, and wirelessly powered. These devices negate the need for rigid, brittle silicon electronic boards and allow for intimate skin connection. These flexible electronic sensors can be integrated with medical compression stockings and pair with smartphones for wireless data transmission and monitoring.

In embodiments, provided are pressure sensing systems and methods for adequate therapeutic compression with inelastic and elastic compression stockings of the legs and arms. The systems and methods may also assess patient adherence of their use of inelastic and elastic compression stockings. In embodiments, the systems and methods provide the ability to track the efficacy of oral medications (e.g. furosemide) used to reduce leg edema in other conditions such as heart failure and/or assess swelling in the setting of acute thrombosis of the lower legs. Further, some embodiments provide the ability to determine when a compression stocking has lost tensile strength reminding users/physicians to purchase a new device

Provided is a thin, flexible platform allows intimate connection with the skin that allows for continued use. Additionally, sensor design allows for unparalleled accuracy and repeatability of pressure that can be placed at anatomically important locations of the leg. Another advantage of the provided systems and methods is a lack of rigid electronics, which reduces risk of skin infection or inadvertent injury. The provided communications system (e.g. near-field communication, Bluetooth) enables wireless data transfer to standard smartphones. Some embodiments utilize temperature sensing, which is useful to determine patient adherence in regards to wearing compression garments and may be tracked by a patient's own smartphone or the device itself. In some embodiments, the smart compression stocking may allow for disposal when the compression provided by the garment is no longer of therapeutic value.

This example provides a compression stocking which incorporates a pressure sensor (capacitive or piezoresistive), integration of this pressure sensor within a flexible, electronic platform which further includes a communication system (e.g. a near-field communication chip). Some embodiments may also include a temperature sensor and/or an adhesive backing. The described garment may allow for integration with a smartphone enabling data download and off-line evaluation of continuous pressure data. This data can trigger text messages that allow users to increase compression as needed for therapeutic benefit.

In embodiments, LED integrated onto the flexible, electronic platform may provide notification of pressure thresholds enabling immediate user feedback when a level of pressure falls below therapeutic levels or reaches a pressure level that may increase the risk of ischemia.

Additionally, some embodiments sense leg volume (e.g. through the use of a bioimpedance measurements or strain gauges) allowing for the determination of the optimal balance between compression strength (interface pressure) and edema reduction (volume).

The provided pressure sensor serves as the basis of measurement. The pressure sensor is embedded within a flexible, electronic platform which may include an adhesive backing for integration with a therapeutic garment. The electronic platform includes thin wires organized in serpentine fashion to allow for skin deformations without loss of performance. A communication system is integrated within these wires and collects data longitudinally from the pressure sensor. In some embodiments, this negates the need for an onboard battery. The pressure sensor, or an additional sensor, may also be able to detect skin temperature, which can be used to assess whether a patient is using their compression garment.

In an embodiment, device is applied directly to the skin of the lower legs at key anatomical locations (overlying the gastrocnemius muscle, the medial ankle, and upper anterolateral thigh). The overlying compression stocking is applied over the sensor. Given the thinness of the device, the device will be mechanically invisible to the user. In additional embodiments, the flexible electronic device is integrated within the compression stocking, allowing for application to the skin simply by putting on the garment.

The sensor's data is then transferred onto a smartphone for data collection through the communication system. The data can be used to send text messages to inform the user to increase the tightness of fit of the compression garment in order to increase interface pressure. These triggers can be programmed by the physician.

The completed prototype will require no external wires or rigid circuit boards. The devices and systems described herein may be disposable. In an embodiment, the data from sensor will be stored on the communication system within the platform. The user will simply pair any smartphone to capture the data. As a test, pressure was applied using a standard sphygmomanometer, illustrating the differences in pressure sensed by the device.

The ability to seamlessly measure and wirelessly transmit critical interface pressure data greatly augments the ability to care for patients suffering from VSUs. Beyond VSUs, medical compression stockings have demonstrated clinical benefit for other lower leg issues including vein varicosities and deep vein thrombosis. The described exemplary device represents a major innovation in the field of medical compression stockings.

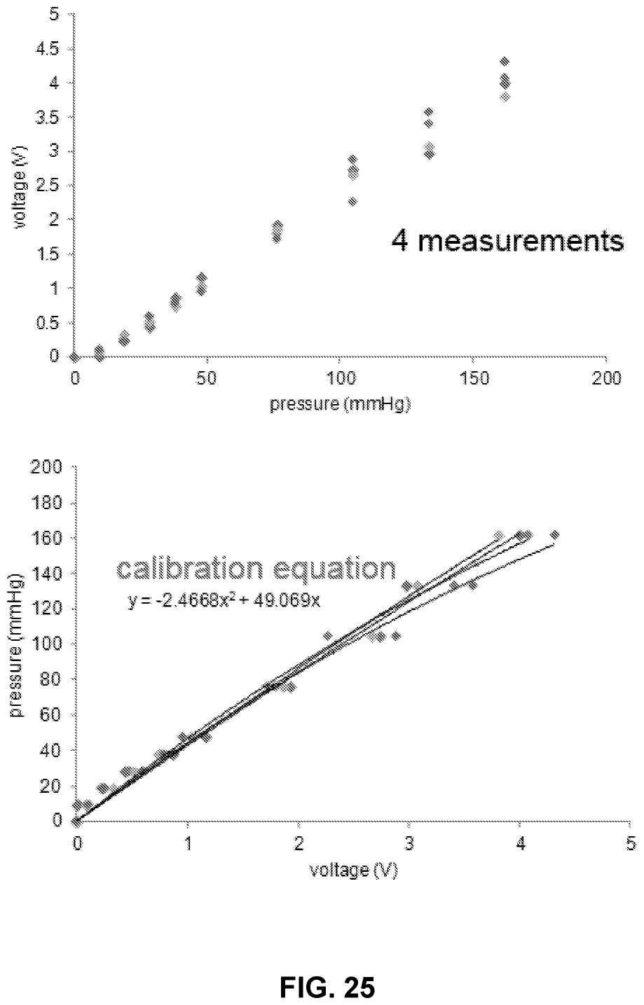

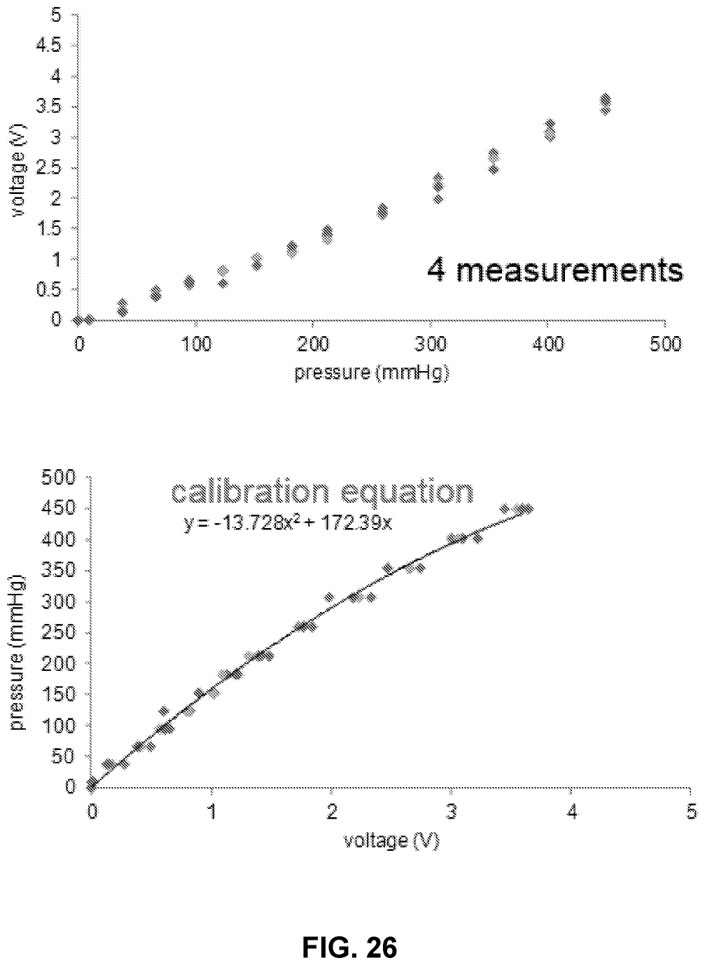

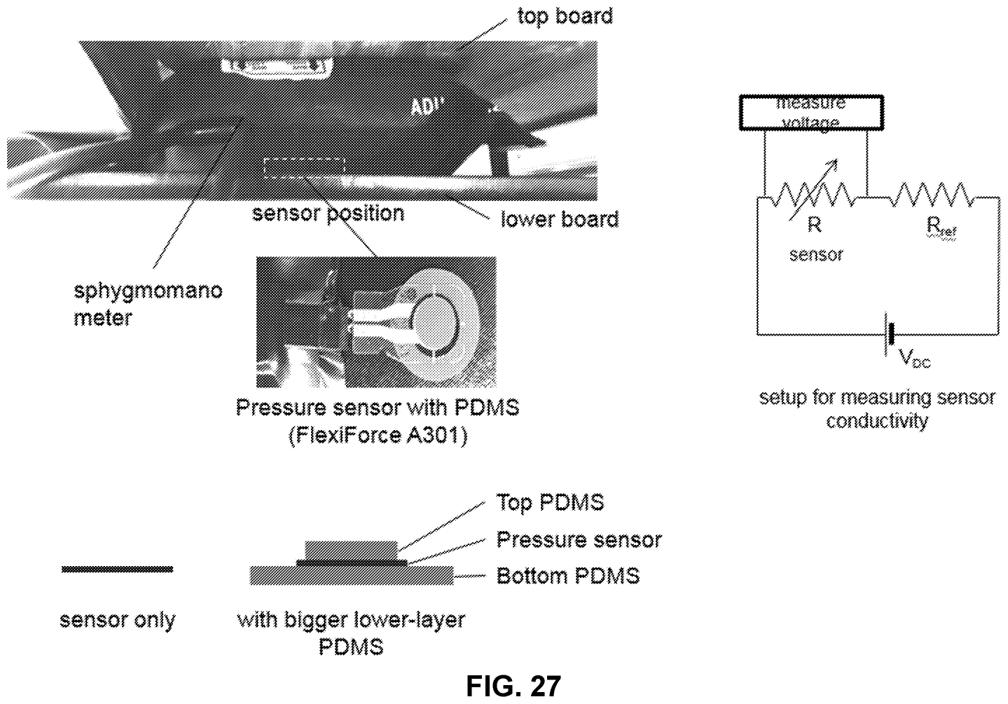

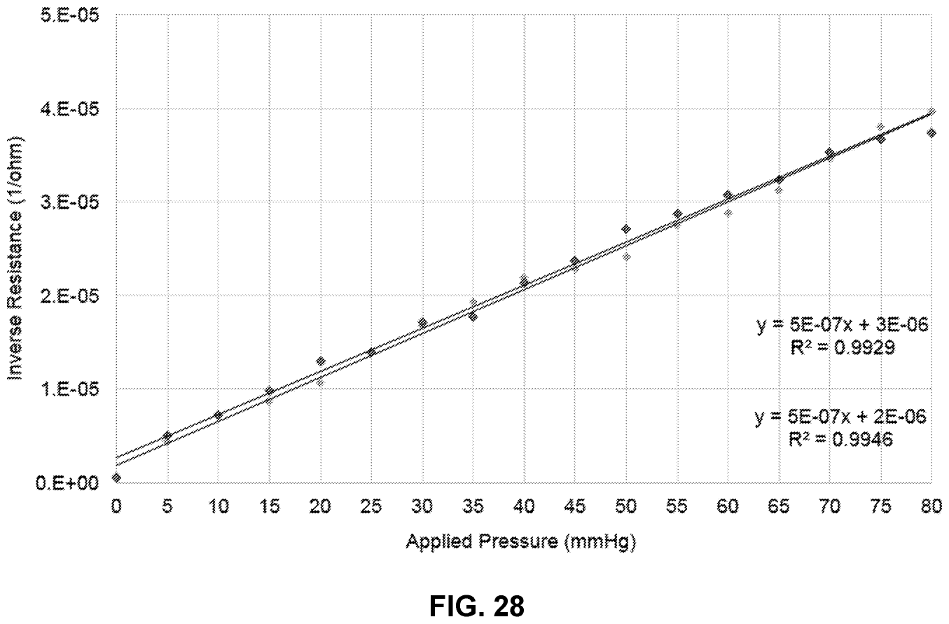

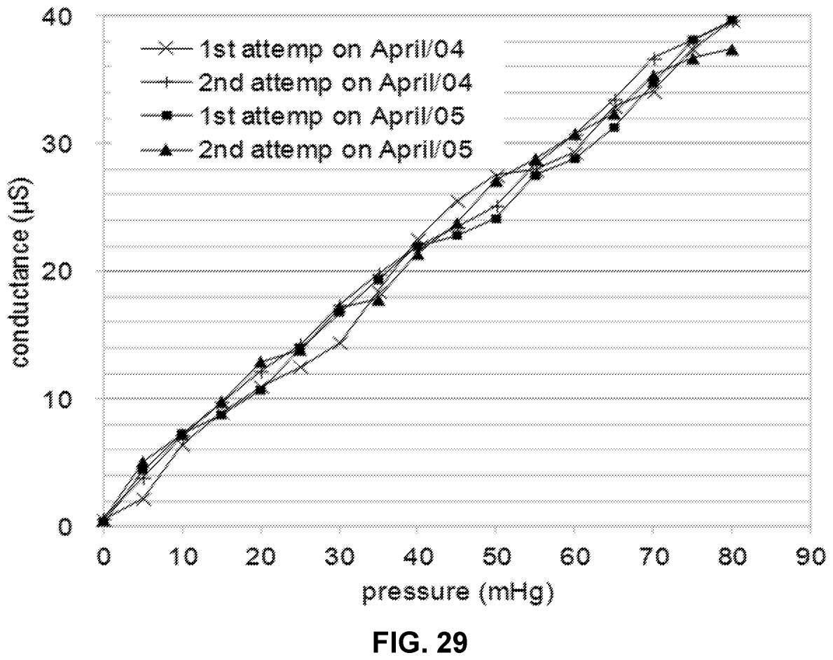

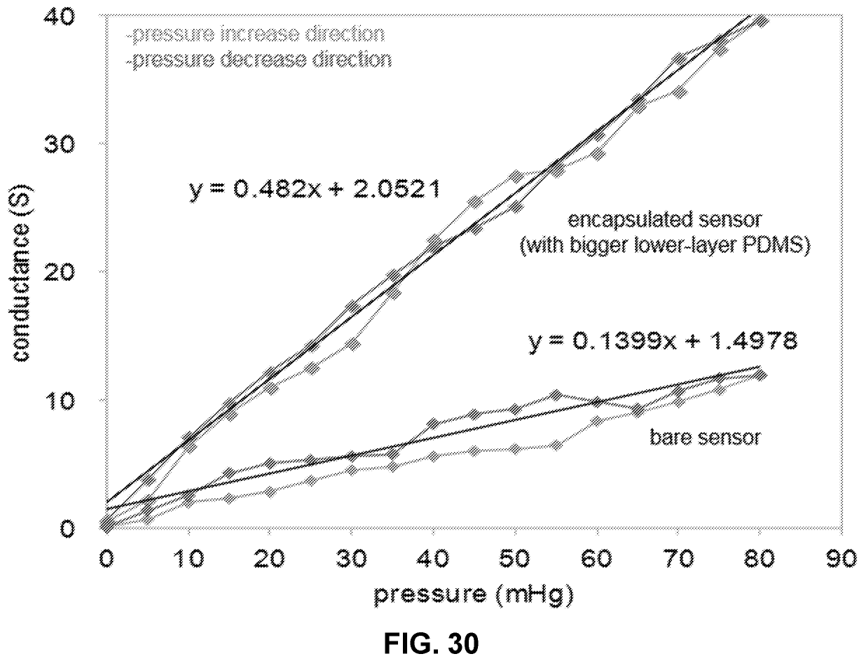

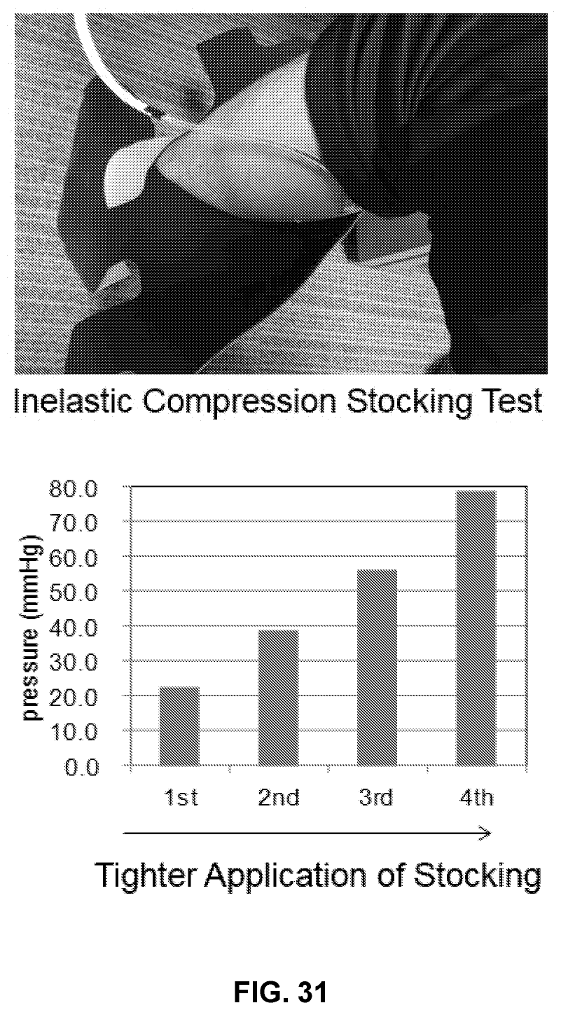

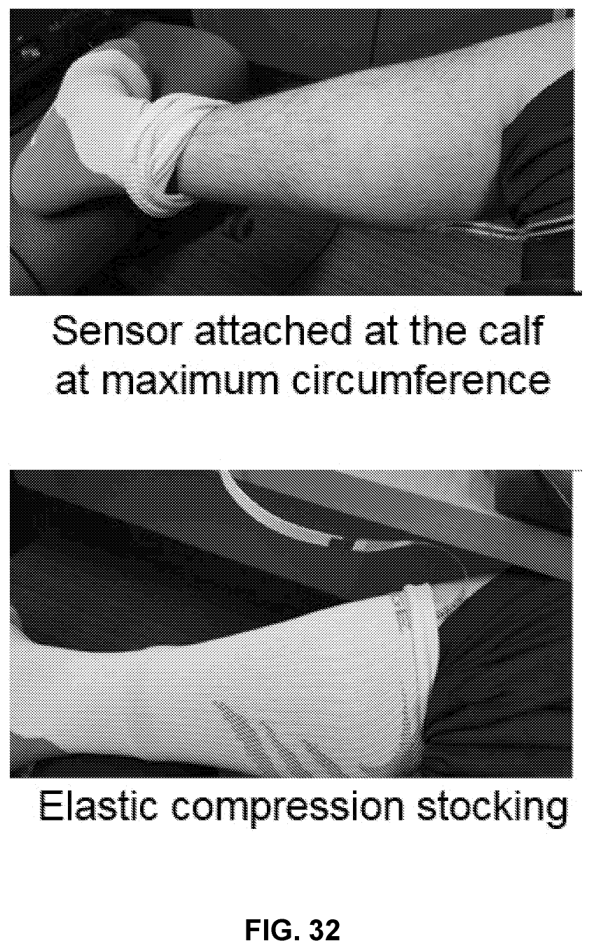

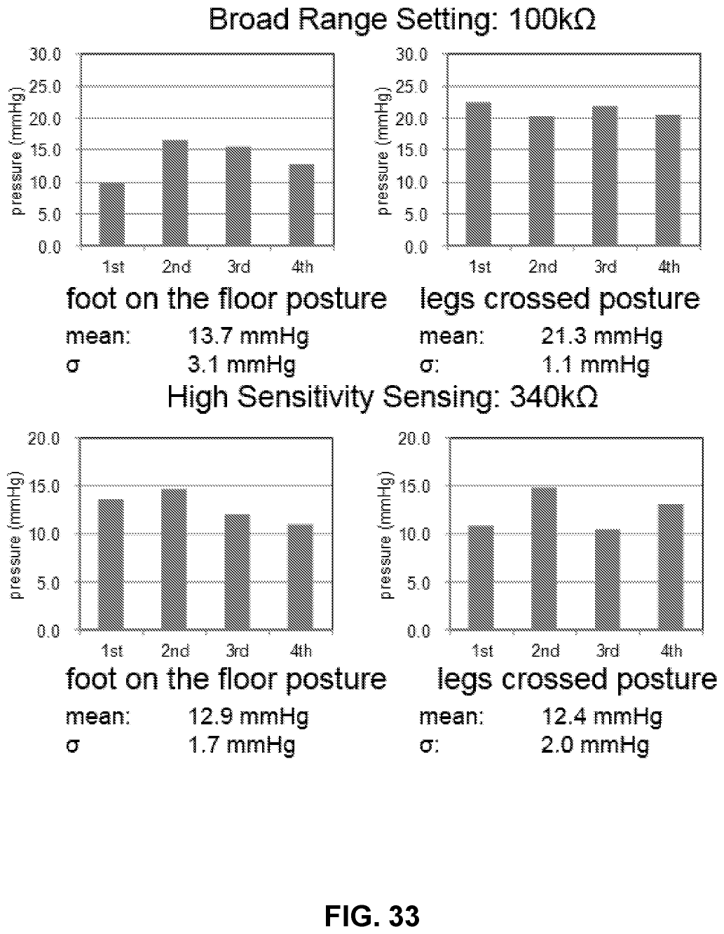

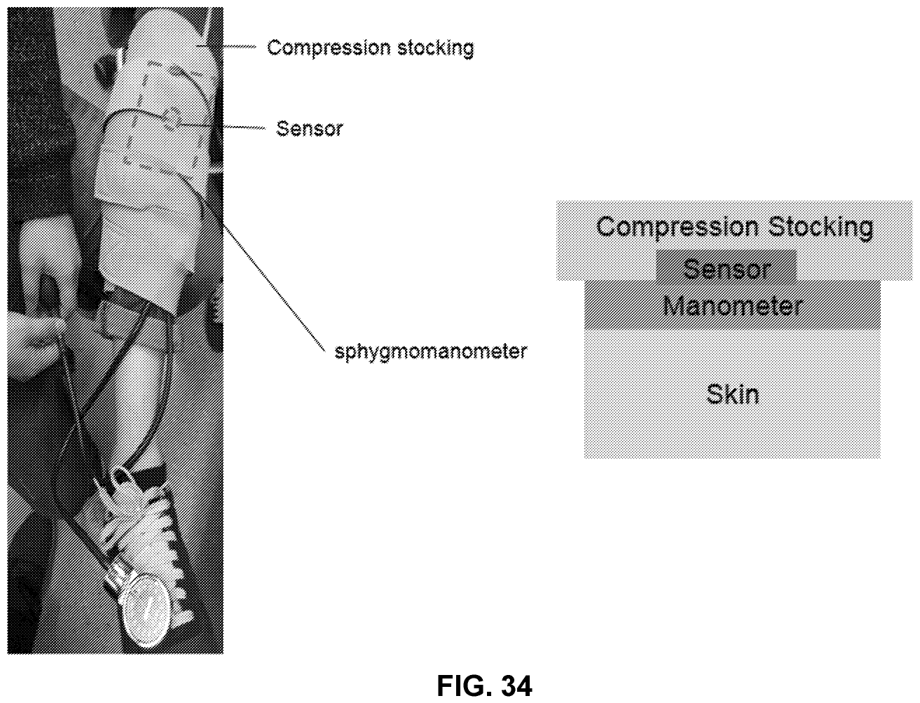

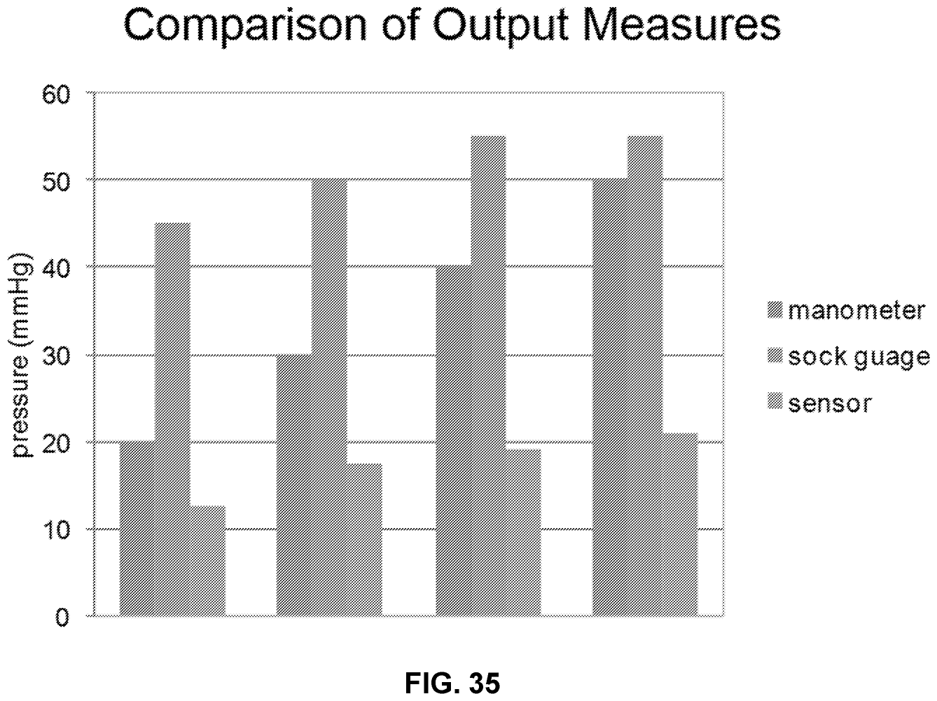

Calibration of the functional fabrics and sensors described herein plays an important role in providing therapeutic benefits and are illustrated in FIGS. 25-28. The calibration procedure for the pressure sensor is standardized, and consistent where the graduated addition of pressure reflects real-world operation of a therapeutic compression garment.

### Example 2: Compression Garments for Measurement of Limb Volume

Beyond interface pressure, measurement of limb volume also holds significant clinical value. The measurement of limb volume, for example leg volume, is commonly performed by modeling the leg as a cylinder and determining volume based on circumference with tape measures. Hence, the measurement of leg volume is labor intensive and lacks sensitivity to small but still meaningful changes in leg volume. Furthermore, tape measure assessments of leg volume do not provide an understanding of the nature of edema.

A first example method of assessing leg volume is the use of bioimpedance. Bioimpedance provides a measurement of passive electrical properties that can be used to characterize biological tissue. By measuring the impedance of tissue in response to stimulatory currents, and voltages of varying frequencies, predictions can be made of a limb's fluid status or ischemic state. In the case of leg edema, the excess extracellular fluid leads to changes in the electrical impedance of the tissue. Measurement of bioimpedance is cost-effective, non-invasive, repeatable, and sensitive.

In embodiment, bioimpedance is utilized to measure limb volume. Bioimpedance provides a measurement of passive electrical properties that can be used to characterize biological tissue. By measuring the impedance of tissue in response to stimulatory currents, and voltages of varying frequencies, predictions can be made of a limb's fluid status or ischemic state. In the case of leg edema, the excess extracellular fluid leads to changes in the electrical impedance of the tissue. Measurement of bioimpedance is cost-effective, non-invasive, repeatable, and sensitive.

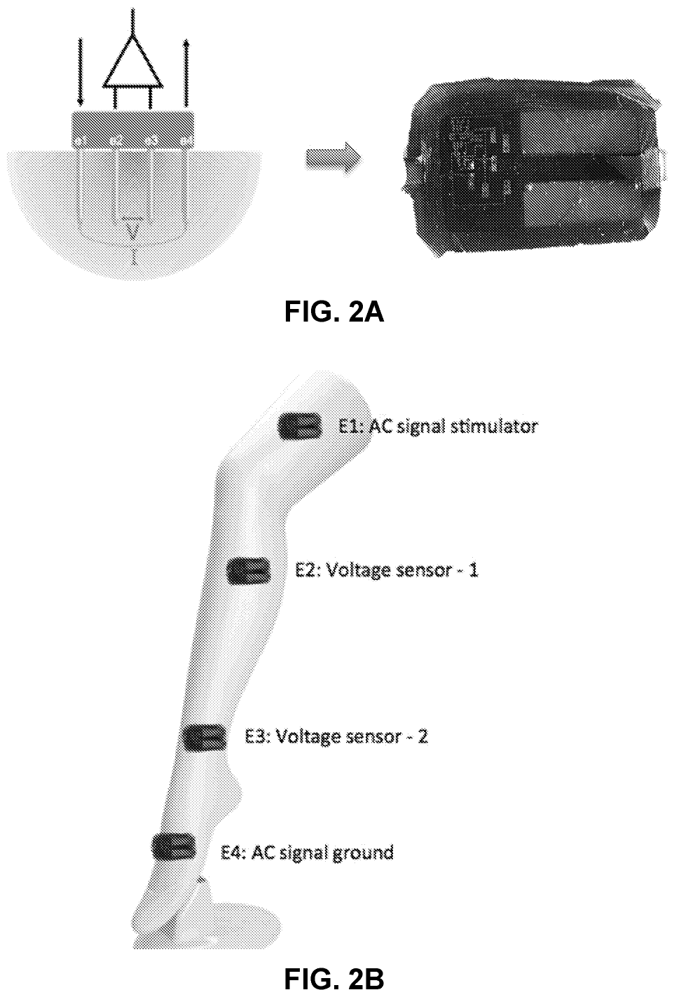

The use of bioimpedance to assess biological phenomena is well established. Those skilled in the art will know that standard ECG electrodes (electrochemical electrodes of Ag—AgCl) or metallic plunge probes (platinum, gold, or stainless steel) can be applied on the tissue surface in order to determine bioimpedance. Another method of determining bioimpedance involves a tetrapolar impedance measurement system. This reduces the contribution of motion artifact and measurement of impedance associated with the skin-sensor interface. Example tetrapolar impedance systems are provided in FIG. 2. AC signal is driven through two electrodes (e1 and e4). The other two electrodes are then used to detect the voltage drop (e2 and e3).

The placement of the various sensors is important in the assessment of limb volume. We employ standard anatomical locations (area of maximum girth of the gastrocnemius muscle or insertion of the gastrocnemius muscle at the Achilles tendon) to ensure a normalized distance for patients of varying leg lengths.

The frequency and current of the AC signal is selected to minimize the risk of patient harm. Some embodiments utilize frequency ranges of 5 to 100 kHz for assessing biological tissue volume. In an embodiment, 50 kHz frequency is used.

Bioimpedance Method 1 (FIG. 2A): In this embodiment, a tetrapolar impedance system is integrated within each individual flexible electronic sensor with a pressure sensor, and communication system. This enables separate assessments of bioimpedance at various locations of the leg that are of anatomical importance. E1 will deliver the stimulatory AC signal with e4 serving as the ground. E2 and E3 act as the differential sensor to detect voltage drop. In an embodiment, the flexible electronic sensors are placed on the anterior thigh, proximal calf, distal calf, and medial malleolus.

Bioimpedance Method 2 (FIG. 2B): In this embodiment, the components of the bio-impedance system will be separated into separate flexible electronic sensors. E1 is a stimulating board which provides AC signal and placed on the upper thigh. E4 provides a ground for the stimulatory AC signal. E2 and E3 are bioimpedance sensing electrodes. These two electrodes sense the differences in interface voltage. E2 may be placed more proximally on the leg while E3 may be placed distally on the leg. The differential in voltage can be extracted from these sensors.

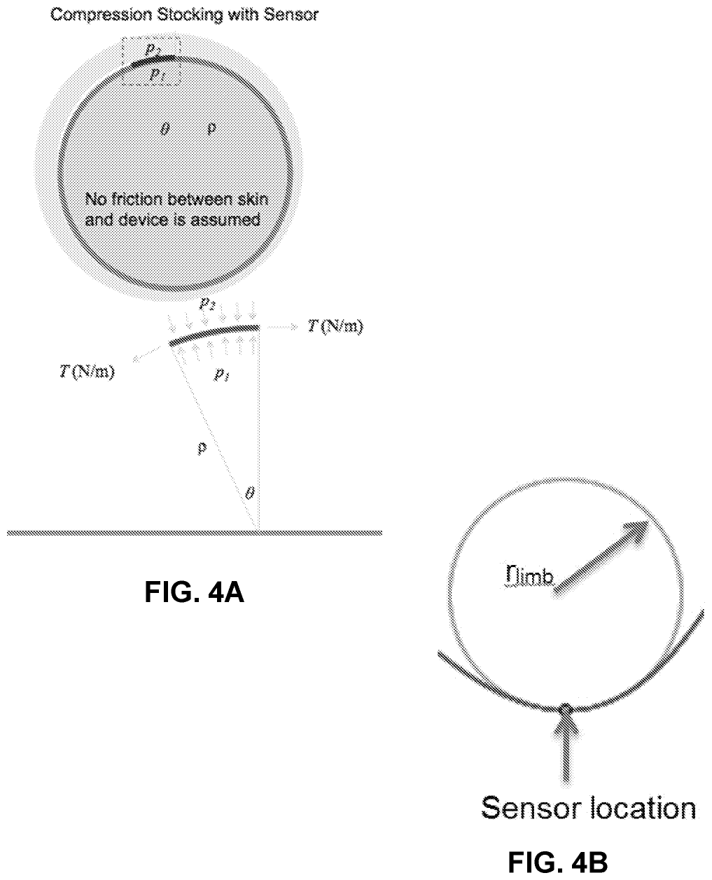

A second example method of determining leg volume is through modeling the lower leg as a cylinder. As described in FIG. 4, we model the sensor within a system that includes the compression stocking and leg (modeled as a cylinder). The pressure exerted by the stocking (p₂) is assumed equivalent to the opposite pressure exerted by the leg (p₁). Another strain gauge sensor wrapped circumferentially around the leg can be employed to sense changes in tension (T).

With pressure and tension measurements, the radius of curvature (p) can be derived using the following equation:

pρ=T₁ Eqn. 1

Assuming that the radius of curvature at the location of the sensor is tangential to the lower limb, then the radius of curvature calculated by the sensor would equal the lower limb radius (r_{limb}).

The volume of the limb can be determined using this derived cylinder radius and the length of the limb, which is a fixed value:

V=π*r_{limb}*limb length Eqn. 2

Since limb length is fixed, changes in r_{limb} can be used to express leg volume changes.

### Example 3: Epidermal Interface Pressure Sensor (EIPS)

In this example, provided is a soft, flexible device platform containing a pressure sensor. In some embodiments, the device platform exhibits a Young's modulus comparable to natural skin. In some embodiments, the device is encapsulated in PDMS to reduce contract stress, protect sensors and communications system on the device and improve sensor performance, for example, piezoelectric pressure sensor performance.

In some embodiments, the provided systems or methods are “mechanically invisible” or “user invisible.” User invisibility is an important consideration for sensors designed for long-term use. In patients with medical problems such as lower leg edema or chronic venous insufficiency, the skin is particularly sensitive to irritation, susceptible to injury, and prone to infection. Thus, user invisibility is an important medical consideration. The determination of user invisibility can be achieved in two ways. From a quantitative method, user invisibility can be determined by measuring skin temperature. When skin temperature exceeds a certain temperature, the sensor can alert the user for the need to potentially remove the device. Other onboard warning methods may include assessments of blood flow or skin redness, which may also indicate the need for sensor removal.

The user can also be asked to respond to directed questions via SMS text messages or mobile phone application prompts. The user can indicate if they are experiencing any pain, irritation, itching, or redness related to the device at specified time intervals.

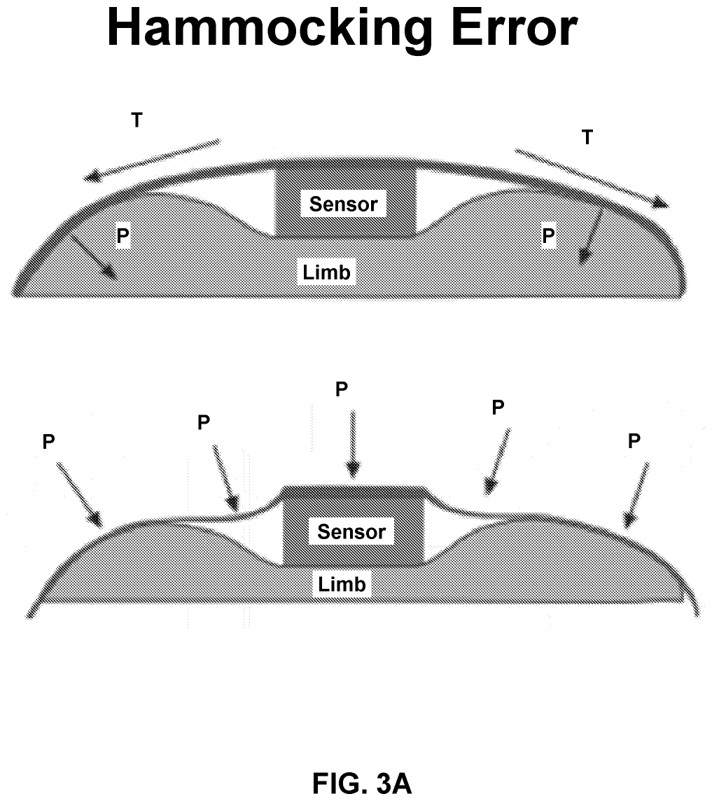

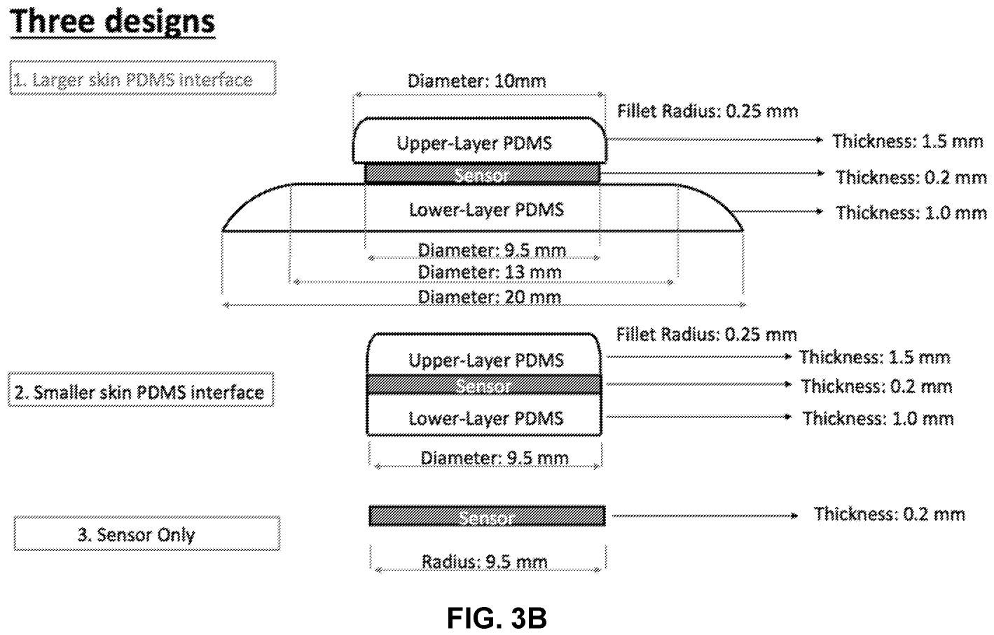

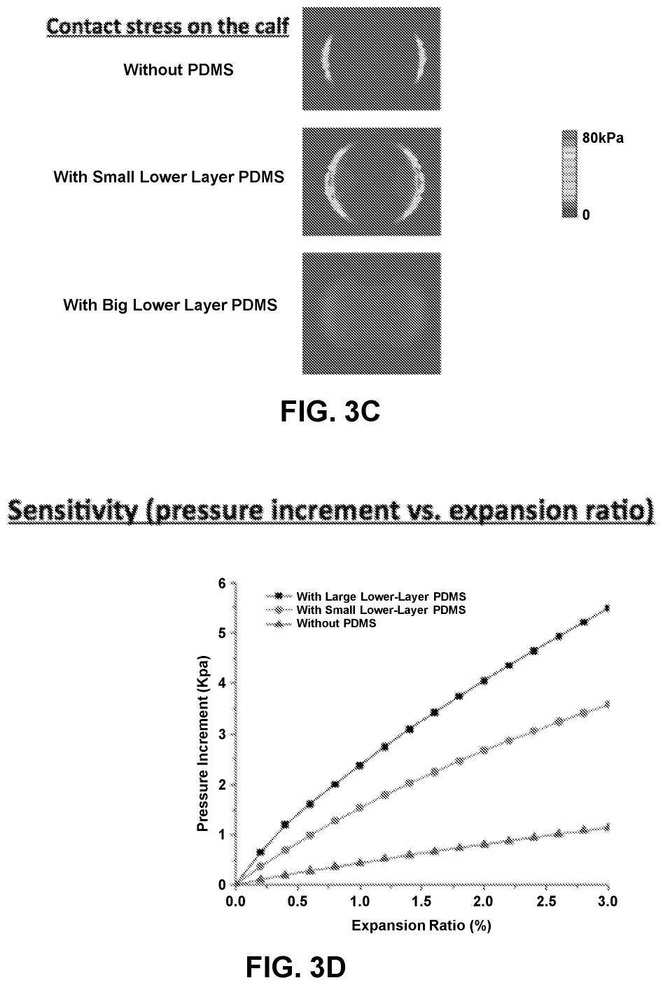

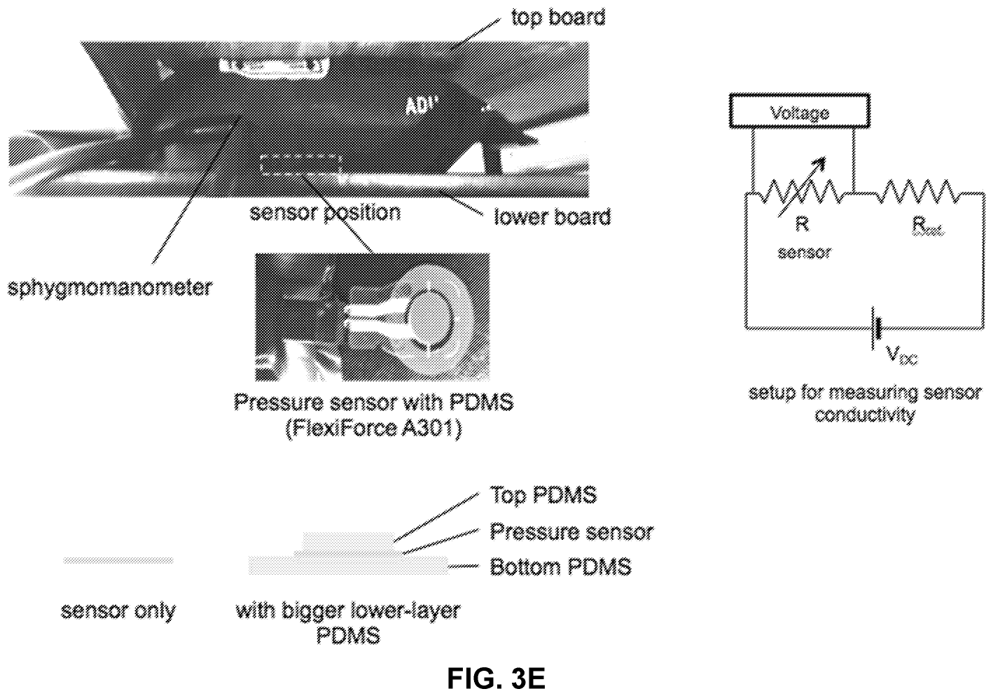

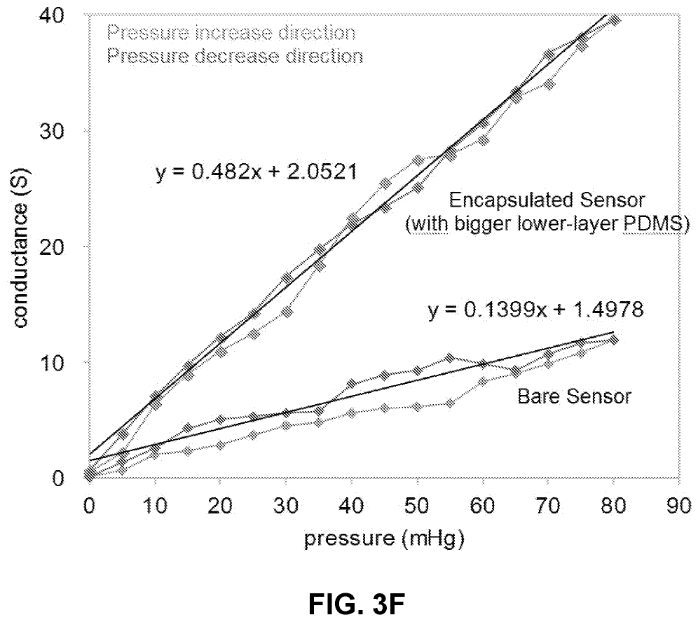

Achieving a small sensor profile directed by in-depth mechanical modeling ensures accurate sensing. A key problem with interface sensors for pressure is hammocking error (FIG. 3A). Hammocking error may occur when a sensor exhibits significant modulus differences from skin at the interfaces between the sensor and the skin, non-natural pressure signals occur. Hammocking error may be reduced by placing a larger, thin layer of PDMS on the side of the device in contact with the skin (FIG. 3B). Due to this layer of PDMS, there is less discontinuities in modulus that leads to aberrant results.

We modeled two different encapsulation methods compared to the bare sensor based on their inherent mechanical properties. Our modeling indicates that an encapsulated sensor with a larger PDMS skin interface yielded the best mechanical results as illustrated in FIG. 3C by finite element modeling. Encapsulation in this method dramatically reduces and, in some cases, virtually eliminates contact stress.

Experiments were conducted to compare the various sensor configurations described herein, where conductance was measured while using a sphygmomanometer to apply incremental increasing pressures. The sensor was placed between two rigid boards FIG. 3E.

Results demonstrate that encapsulation with a larger PDMS interface with the skin compared to the sensor alone enabled more discriminatory ability in bench testing. As in FIG. 3F, a greater use of the full-scale range of the sensor is employed with the encapsulated sensor. Furthermore, there is a decrease in hysteresis as well. High linearity (R²>0.98) was achieved.

### Example 4: Novel Clinical Metrics

The ability to accurately measure and store interface pressure along with other important biometric data (temperature and leg volume) enables the determination of novel, useful clinical metrics. For example, the sensor can be employed to test fabric failure over time. Fabric failure can be expressed as a change in interface pressure that is supplied by the compression stocking in different body positions.

In one example, a pressure sensor averages interface pressure at supine position for 30 second. Then, the sensor averages interface pressure at standing position for 30 second. The Difference of the interface pressure is taken to produce a static stiffness index (SSI). If interface pressure difference >20% of manufacturer specifications, then it indicates to user and clinician that a new garment is needed, enabling a simple method to enable testing of fabric failure without the need for specialized equipment.

Another example is a device that measures pressure and limb volume together, allowing for a new clinical index. This applied pressure over limb volume change would represent an individual patient's tissue response to therapeutic compression. Certain individuals may require higher interface pressure to achieve the same edema reduction compared to others. This enables personalized delivery of compression therapy that is adaptive.

Beyond continuous sampling, the provided systems can be modified to provide cumulative pressure sensing. For example, the sensor output can be used to continuously charge a capacitor in the circuit. The total capacitance at the end of a fixed time period can provide a single metric of applied pressure over time or total applied pressure. This would take into account patient adherence and alterations in pressure with body position but summing all pressures sensed throughout a given time period.

The sensor may also be employed to track wound healing over time. Venous stasis ulcers are directly secondary to chronic venous insufficiency (which leads to leg edema). The sensor can incorporate skin temperature as a way to track infection. The sensor can also utilize temperature to sense and quantify blood flow directly to the wound bed, which is important for wound healing.

### Example 5: Epidermal Electronics in Compression Stockings

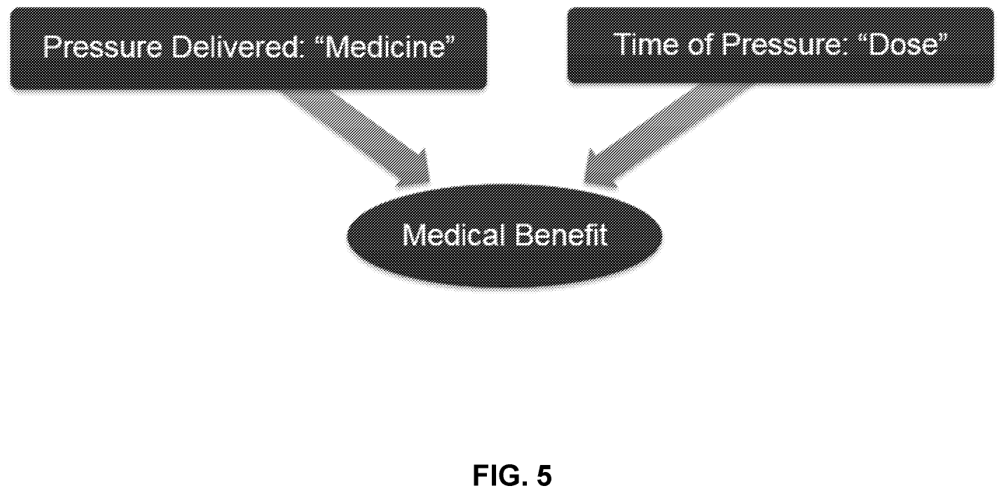

Inelastic and elastic compression stockings are a common treatment for a range of conditions including chronic venous insufficiency, venous stasis ulcers (>35 mmHg improves healing), lipodermatosclerosis, and lymphedema. Treatment incorporates at least two aspects as shown in FIG. 5.

Currently, there are several clinical needs for improved treatment using compression stockings:
1. Need for a continuous measurement system for interface pressure to understand ‘medicine’ of compression
2. Need for assessment of patient adherence and real-world usage to understand ‘dose’ of compression
3. Need for data storage and transmission—ideally wireless
4. Need for integration with existing compression stockings
5. Need for a method to assess garment performance failure

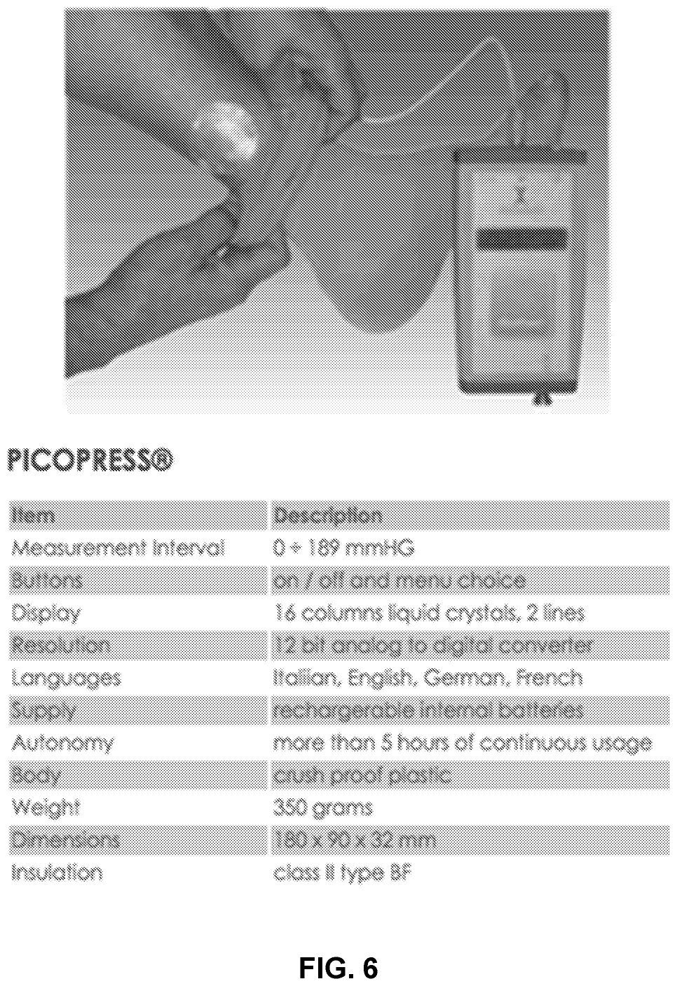

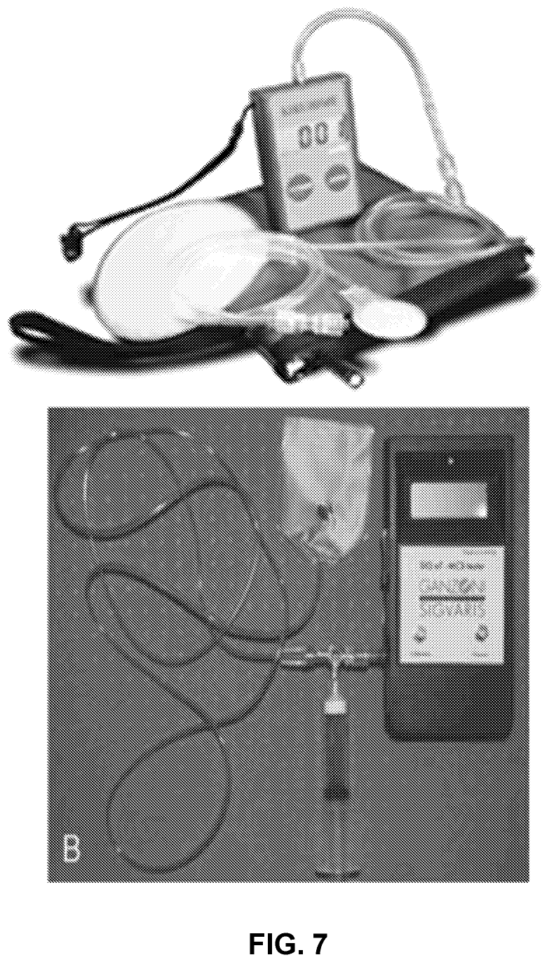

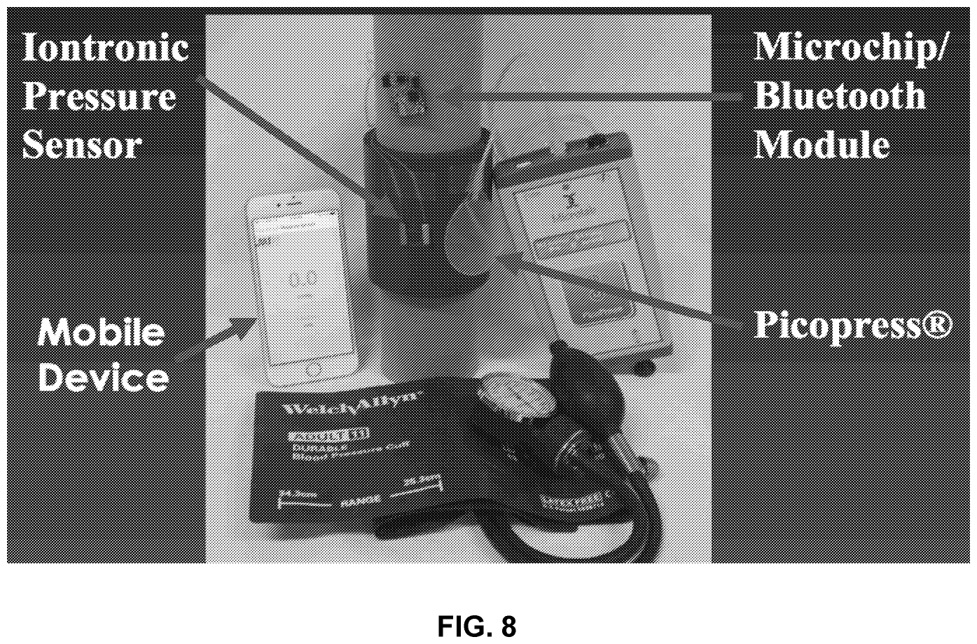

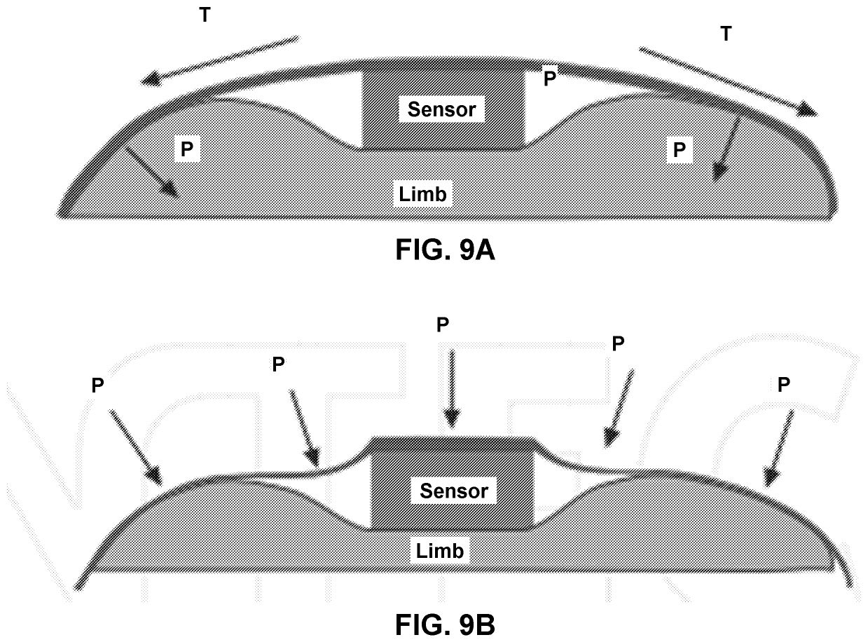

Examples of pressure sensors used in research surrounding compression treatment are provided in FIGS. 6-8.

Academic research and literature has proposed several design criteria which may be important for developing compression stocking devices as described in Table 1. Two potential commercial sensors were compared under the design criteria, also shown in Table 1.

TABLE 1
Proposed design criteria in literature
(source: Derm. Surg. 2006, 32: 224-233)

| | | | EIPS as |
|---|---|---|---|
| Criteria | PicoPress | VenoSense | described herein |
| Reliability | +++ | + | ++ |
| Accuracy | +++ | ++ | ++ |
| Low hysteresis | +++ | + | ++ |
| Thin | − | ++ | +++ |
| Continuous Output | − | + | +++ |
| Accuracy | ++ | + | ++ |
| Variable Sensor Sizes | − | − | +++ |
| Wireless Transmission | − | +++ | +++ |
| Temperature | − | − | +++ |
| Suitable for Daily Use | − | + | +++ |
| Range (0-100 mmHg) | +++ | +++ | +++ |
| Commercially Available | +++ | − | − |

The present example was designed to address the following clinical needs:
1. Need for a continuous measurement system for interface pressure
2. Need for data transmission—ideally wireless capabilities
3. Need for assessment of patient adherence and real-world usage
4. Need for integration with existing compression stocking garments
5. Need for a method to assess garment performance failure
   Which in turn lead to the following technical design criteria:
1. Continuous interface pressure sensing (0-100 mmHg+/−1 mmHg)
2. Provide wireless transmission of onboard sensor data for additional analysis
3. Provide onboard temperature sensing to quantify pressure sensor
4. Presents no risk to skin irritation, or skin injury using a flexible, epidermal interface with at least 1 week of use
5. Enable straightforward integration with any existing compression garment
6. Less than 20% error vs. gold-standard pressure sensor (PicoPress) across the full range of the device
7. Enable unique identification of patient information
8. Provide method for onboard testing of compression stocking performance

Intrusive rigid sensor causes deformation of the limb and/or the stocking in what is known as hammocking error, which is described in FIG. 9. Intimate skin connection between the sensor, stocking and limb increases both sensing accuracy and user acceptance.

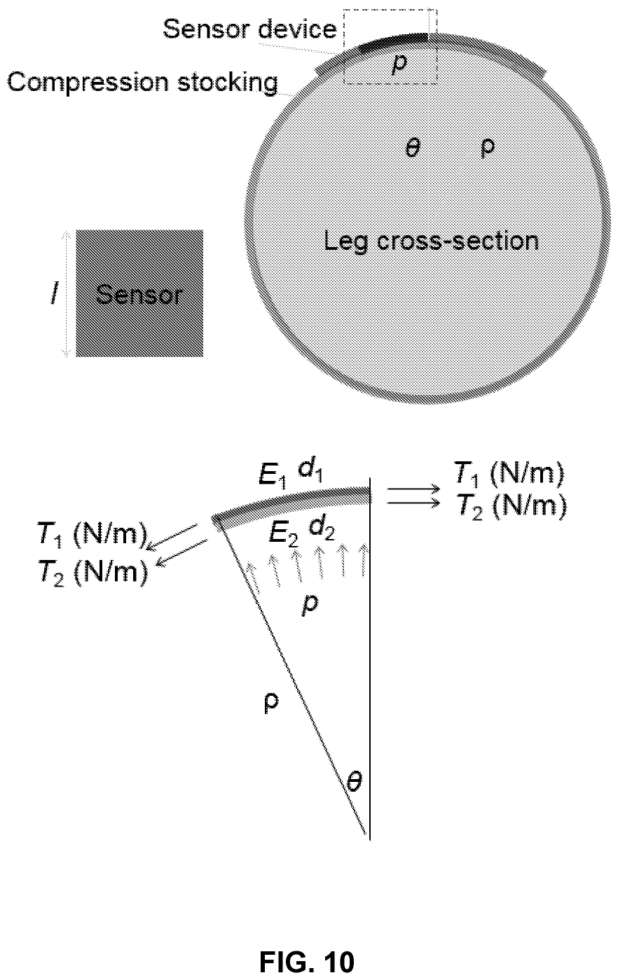

The physics of the sensor, stocking and limb can be described physically in FIG. 10 and by Equation 1:

pρ=T (Eqn 1)

As previously described, where

T=T₁+T₂ (Eqn 3)

Assuming linear elasticity, the compatibility equation becomes:

$$\frac{{T}_{1}}{{E}_{1} {d}_{1}} = \frac{{T}_{2}}{{E}_{2} {d}_{2}} \qquad ( Eqn 4 )$$

If the flexural rigidity of the device is far smaller than the value of the compression stocking then:

$$p ρ = {T}_{2} ( 1 + \frac{{E}_{1} {d}_{1}}{{E}_{2} {d}_{2}} ) \qquad ( Eqn 5 )$$

where E₁d₁<<E₂d₂, then:

$$\begin{matrix}p ρ ≅ {T}_{2} ; & ( Eqn 6 ) \\ ɛ = \frac{p ρ}{Ed} ; and & ( Eqn 7 ) \\ p = \frac{Ed ɛ}{ρ} . & ( Eqn 8 )\end{matrix}$$

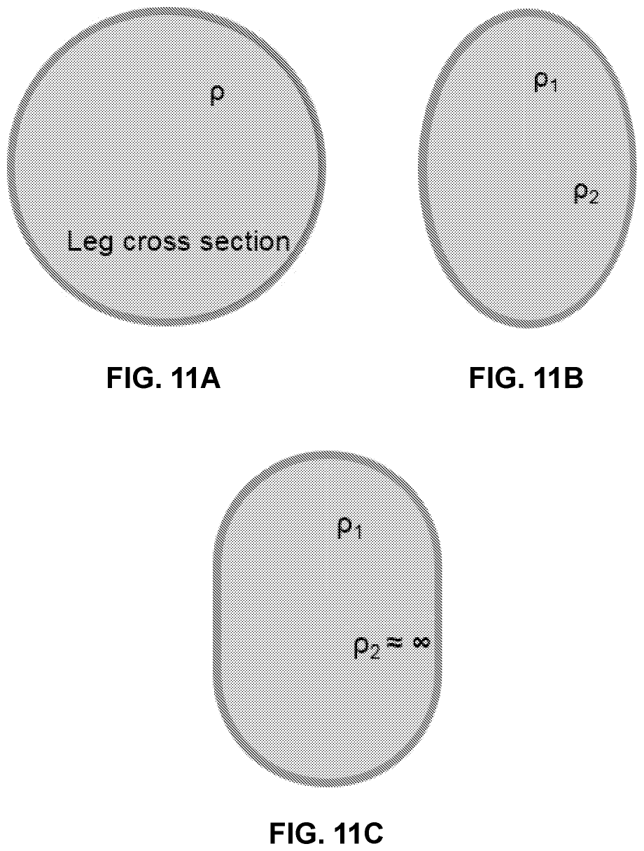

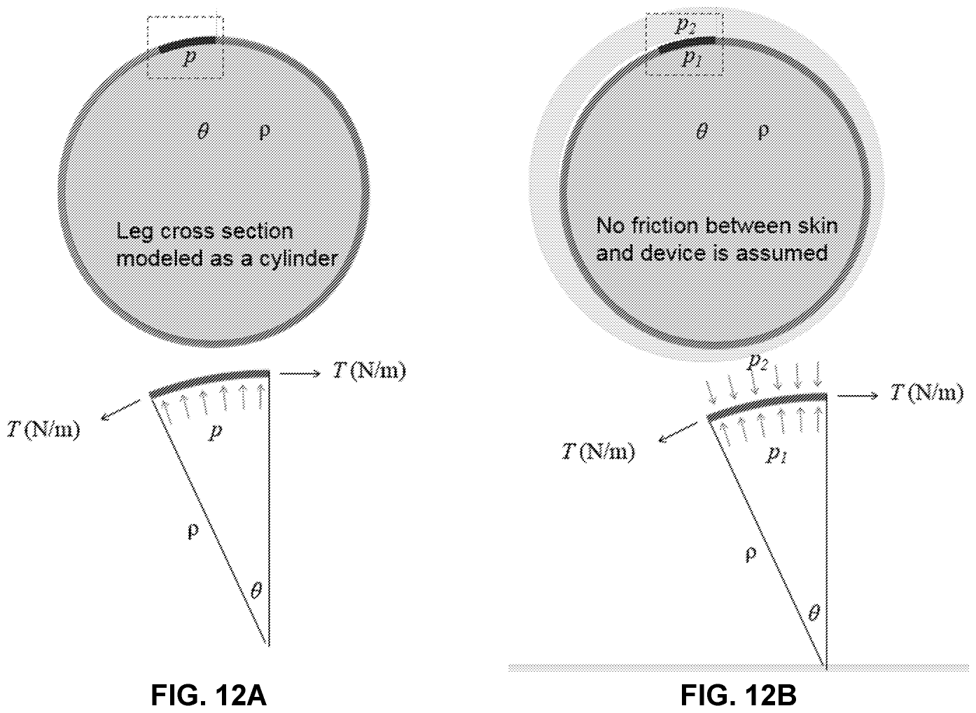

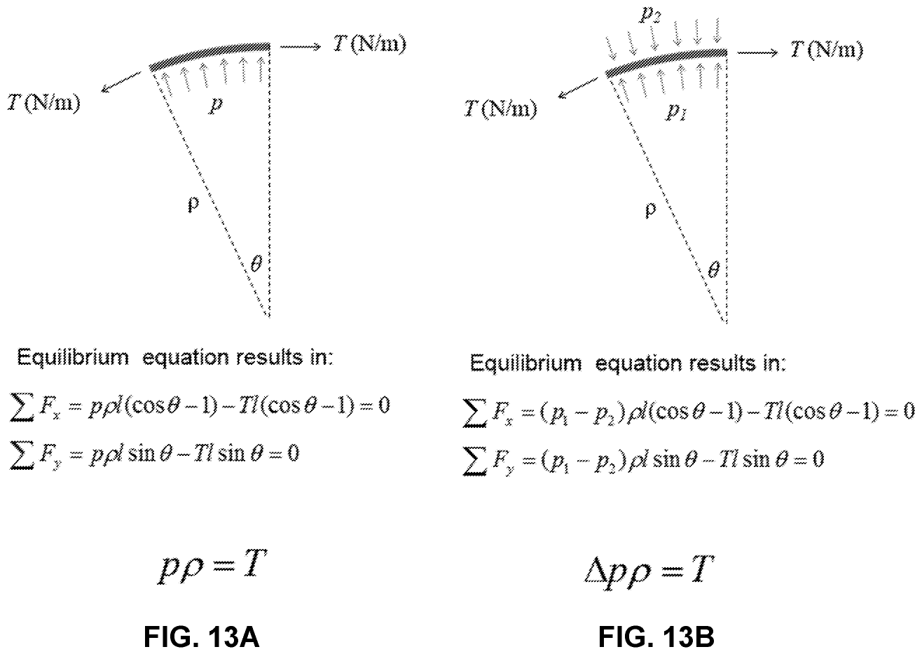

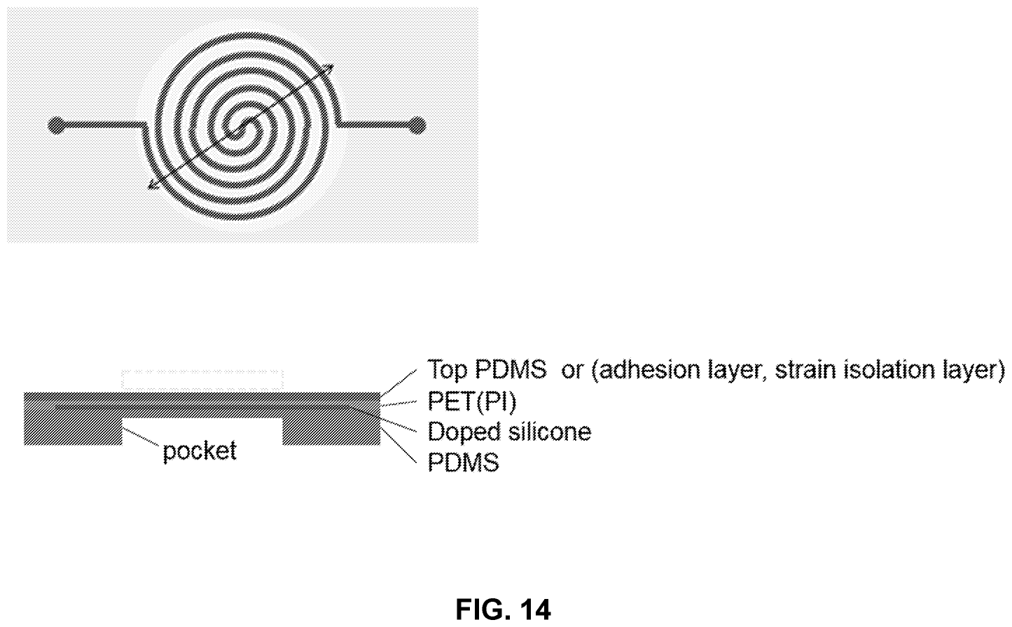

These variables and relationships are illustrated in FIG. 10. Further, the pressure provided by the compression stocking is proportion to the strain of the compression stocking and inversely proportional to the radius of curvature as shown in FIG. 11, assuming that the friction between the skin and the stocking and the motion of muscle are not modeled. FIGS. 12 and 13 provide a comparison of the physical interaction between the compression stocking and the limb both with and without a sensor.

Encapsulating the wireless device and/or sensors in two layers of polyester film on which silver and/or a layer of piezoresistive ink will improve the accuracy and durability of the sensors (this configuration may be referred to herein as FlexiForce). This encapsulation is cost effective, promotes accuracy and linearity but has potential problems with repeatability, hysteresis and strain isolation. To address these issues, we have used three strategies: 1) reduce the thickness of the wireless device/sensor to less than t3 mm to reduce hammocking error; 2) strain isolate the sensor to improve signal stability and 3) replicate the modulus and strain of the sensor with than of skin to achieve mechanical invisibility and ensure patient acceptance.

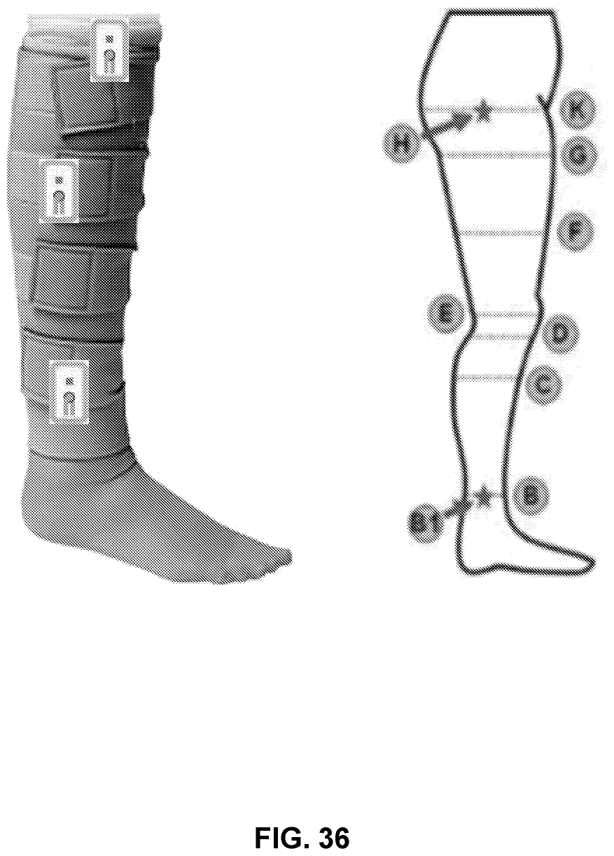

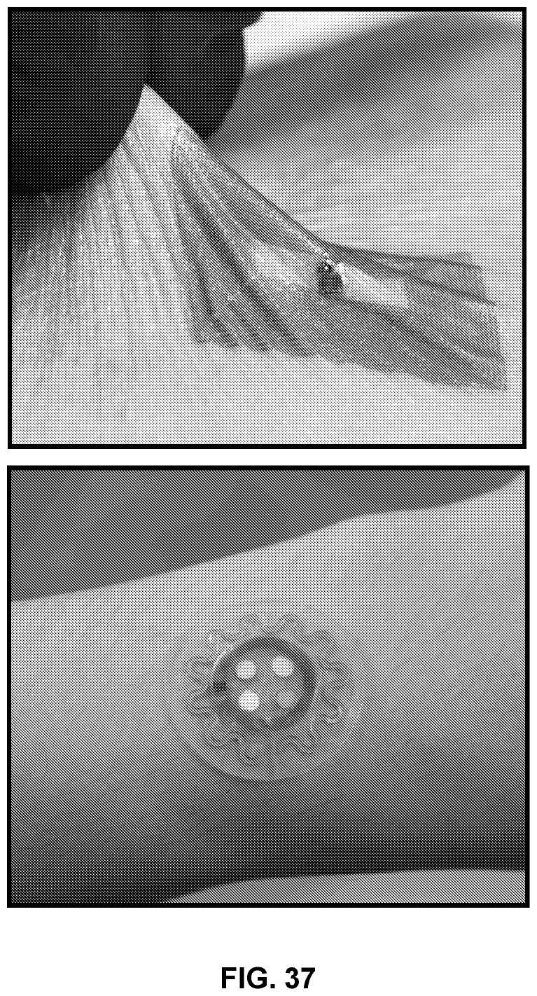

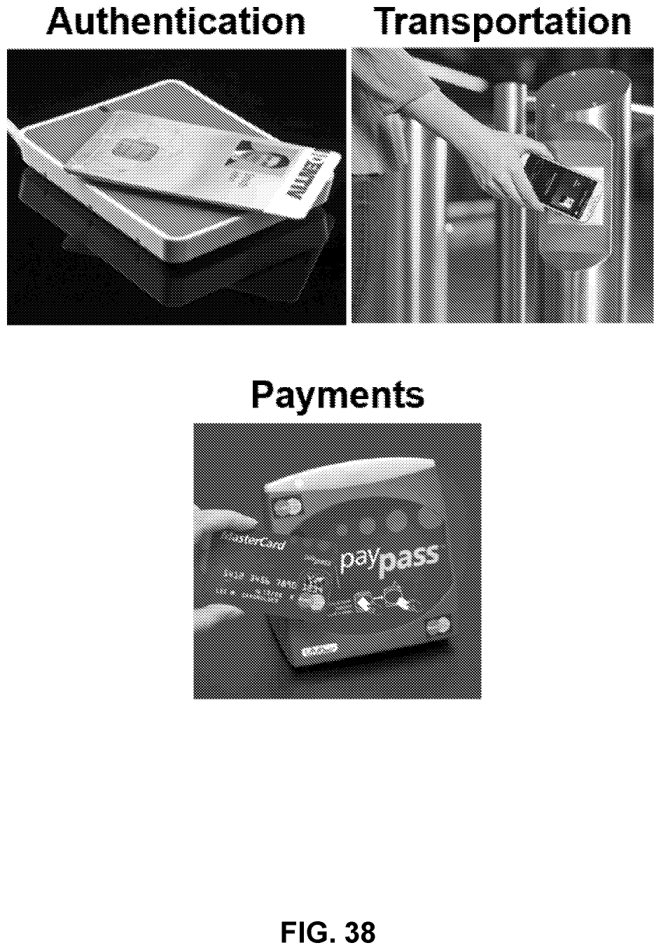

An example compression stocking kit with a plurality of wireless devices and important positions is show in FIG. 36. The positions described in FIG. 36 are given in Table 2:

TABLE 2
Positions shown in FIG. 36

| | |
|---|---|
| B | Ankle at minimum point of girth |
| B1 | Area where Achilles tendon changes into calf muscle |
| | (approx. 10-15 cm proximal to medial malleolus) |
| C | Calf at its maximum girth |
| D | Just below the tibial tuberosity |
| E | Center of the patella and over the back of the knee |
| F | Between K and E (mid-thigh, between patella and groin) |
| G | 5 cm below the center point of the crotch |
| H | Greatest lateral trochanteric projections of the buttock |
| K | Center point of the crotch |

An advantage provided by the systems, methods and devices described herein is that a status stiffness test can be performed at set time intervals (e.g. every three months) to determine if the garment is still effective or if it has failed. These garment tests may be performed by a health professional such as a nurse or a medical assistant. An example of a stiffness test is: 1) Sensor averages interface pressure at supine position for 30 seconds; 2) Sensor averages interface pressure at standing position for 30 seconds; 3) Use the difference between the interface pressures take to produce a static stiffness index; and 4) If the interface pressure difference is greater than 20% of the manufacturer specifications, the indicate to the user and clinician that a new garment is needed.

We have developed a garment in the form of a compression stocking with the following properties:

| Design Consideration | Property Achieved |
|---|---|
| Continuous Pressure Sensing | 0-100 mmHg |
| Wireless Transmission | NFC |
| Onboard Temperature Sensing | NFC |
| Thin, flexible PDMS | Less than 5 mm thickness |
| Integration with Existing | Can be applied to skin |
| Compression Stockings | or cotton liner |
| Enable unique identification | NFC |
| of patient | |

### Example 6—Wireless Stretchable and Bendable Pressure and Temperature Sensors

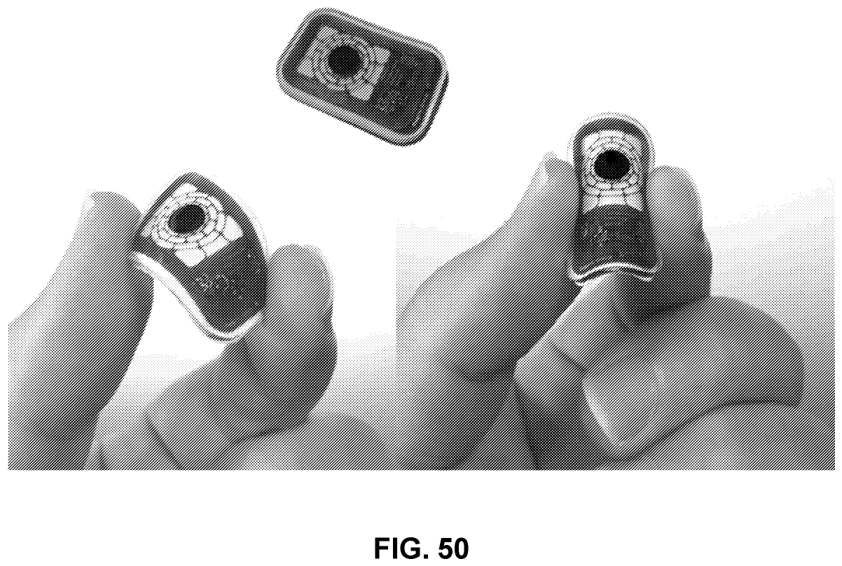

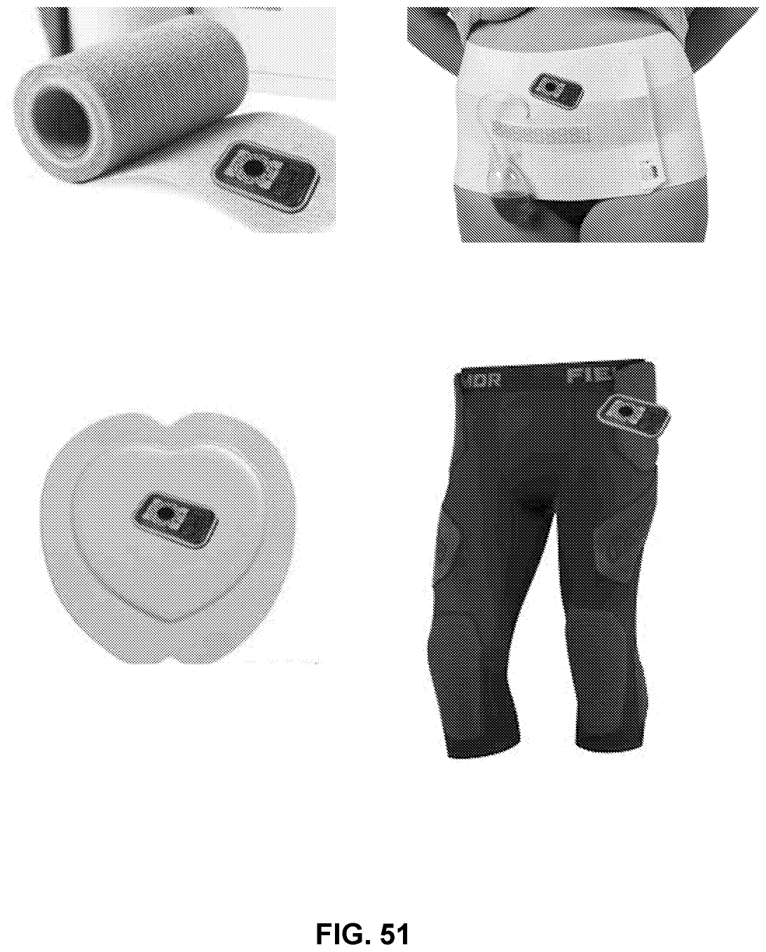

An integrated wireless, wearable temperature sensor is provided in FIG. 50. The sensor is soft and bendable, which facilitates safe skin coupling in high-risk areas prone to wounding. The geometry presented here is rectangular with beveled corners. Other geometries include but are not limited to discoid or ovaloid in shape.

Medical Grade Temperature Sensor: Described herein is the addition of a medical-grade temperature sensor capable of accuracy of 0.1° C. This temperature sensor is thermally isolated from the external environment. The ability to sense cutaneous temperature is an important parameter in assessing local inflammation and wound healing. Elevated cutaneous temperature suggests inflammation and impending wound. Thus, the incorporation of a medical grade temperature with a pressure sensor enables advanced functionalities and the generation of novel clinical metrics relevant to wound healing.

Novel Clinical Metrics: The ability to predict wound healing with prognostic certainty is largely depend on physician visual inspection. Thus, tools capable of providing object measures of wound healing potential would be of high clinical value. In addition, the ability to detect skin inflammation indicative of the formation of a wound prior to clinical manifestation would also be of high interest—this would enable early intervention prior to formation of a significant wound.

The devices and systems described herein can combine onboard pressure, skin temperature across time. This enables the creation of novel predictive clinical metrics relevant to wound healing. For instance, a given period of therapeutic pressure over time (e.g. Pressure—Time index) could be set as a new threshold to assess wound closure likelihood that is confirmed by the skin temperature sensor. Alternatively, the skin temperature sensor over time can indicate a lack of response to therapeutic pressure at a given level and then provide an indicator to increase the absolute interface pressure or increase the time of pressure per day.

Further, multiple pressure and temperature sensors may be placed on the human body or human limb. This enables the creation of a pressure/temperature map. This map may be used to then assess for problem areas that warrant further attention in relationship to other sensors on the same body or limb.

Any of the devices described herein may also be integrated with bandages, wound dressings (specifically pressure ulcer wound healing products), abdominal binders, and athletic gear (e.g. compressive pants, shirts, and sleeves).

### Example 7—Embedded Readers to Reduce User Burden and Enable Continuous Pressure, and Temperature Reading

In some of the embodiments described herein, a smartphone or other mobile device (e.g. tablet or laptop) is necessary to power and scan the data from the device. Other embodiments include non-smartphone NFC readers and power systems to reduce user burden.

For instance, the addition of a battery-powered antennae (e.g. clipped to the outside of a compression stocking) would enable continuous sensing of pressure and temperature. An embedded antennae within a mattress would also enable continuous pressure sensing by providing continuous power.

To reduce user burden, other embodiments outside of the phone to power and read the device include a weight scale or a bathroom floor pad plugged into the wall or battery powered. This enables a user to simply step on a bathroom floor pad daily to measure skin temperature and interface pressure that is then transmitted to the cloud.

### Example 8—Wearable Sensor Enabled Telemedicine

The systems, methods and devices described herein may also be useful in a telemedicine-type platform, wherein the wireless sensor/garment provides useful information to a caregiver, such as a medical professional, friend or family member. Not only are the devices and methods useful in diagnostic or therapeutic applications, but can be used for training and rehabilitation. This is reflected in the devices and systems having two-way communication so that information may sent externally for action to a caregiver and commands received by the medical sensor.

Nursing care and home care for wounds is a time-intensive process. The application, removal, and changing of dressing is largely done without standardization. The sensor reported here would potentially be a useful component of a telemedicine/remote monitoring enabled system. For instance, the sensor may detect a decrease in therapeutic pressure between a therapeutic compression garment and the skin. This would trigger a message or indicator through the smartphone to the cloud that leads to a nursing care event (e.g. re-wrapping). Or, the sensor may detect elevated skin temperature indicative of wound worsening or a new infection triggering a notification to a caregiver or healthcare provider.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups are disclosed separately. When a Markush group or other grouping is used herein, all individual members of the group and all combinations and subcombinations possible of the group are intended to be individually included in the disclosure. Additionally, unless otherwise specified, all isotopic variants of compounds disclosed herein are intended to be encompassed by the disclosure. Specific names of compounds are intended to be exemplary, as it is known that one of ordinary skill in the art can name the same compounds differently.

Every formulation or combination of components described or exemplified herein can be used to practice the invention, unless otherwise stated.

Whenever a range is given in the specification, for example, a temperature range, a modulus range, a number range, a pressure range, a physical dimension, a mechanical property or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1-3. (canceled)

4. A system comprising:
a functional fabric;
at least one flexible, wireless device comprising:
a pressure sensor for measuring one or more pressures exerted by said functional fabric on a region of a body of a subject contacted with said functional fabric system;
a wireless communication system; and
wherein said at least one flexible device is positioned in mechanical communication with a surface of said functional fabric; wherein said flexible wireless device has dimensions and physical properties such that the pressure sensor affects the magnitude of said one or more pressures exerted by said functional fabric by less than or equal to a factor of 25%.

5. (canceled)

6. (canceled)

7. The system of claim 4, wherein said functional fabric is a therapeutic or diagnostic garment.

8. The system of claim 4, wherein said functional fabric is a compression garment.

9. The system of claim 4, wherein said functional fabric comprises one or more of an inelastic material, an elastic material, a woven material, a non-woven material, an adhesive, and a knit material.

10. The system of claim 4, wherein said functional fabric is moistened, heated or cooled.

11. The system of claim 4 wherein said functional fabric is a stocking, sock, sleeve, glove, wrap, bandage, hard cast, soft cast, splint or a pneumatic compression device.

12. The system of claim 4, wherein said functional fabric is a therapeutic compression stocking or bandage.

13. The system of claim 4, wherein said at least one flexible wireless device is affixed to said functional fabric.

14. The system of claim 4, wherein said at least one flexible wireless device is affixed to said functional fabric via an adhesive.

15. The system of claim 4, wherein said at least one flexible wireless device is directly or indirectly supported by a region of a body of a subject contacted with said system.

16. The system of claim 4, wherein said at least one flexible wireless device is configured to be supported by the skin of a subject contacted with said system or supported by a fabric or other layer provided on the skin of a subject contacted with said system or supported.

17. The system of claim 4, wherein said at least one flexible wireless device is characterized by an average Young's modulus less than or equal to less than or equal to 1 MPa.

18. The system of claim 4, wherein said at least one flexible wireless device is characterized by an average Young's modulus matched to within a factor of 100 of the Young's modulus of the skin of a subject.

19. The system of claim 4, wherein said at least one flexible wireless device has a net bending stiffness low enough that the device establishes conformal contact with the skin of the subject.

20. The system of claim 4, wherein said at least one flexible wireless device has a net bending stiffness less than or equal to 1 nN m.

21. The system of claim 4, wherein said at least one flexible wireless device has net flexural rigidity of less than or equal to 1×10⁻⁴ Nm.

22. The system of claim 4, wherein said at least one flexible wireless device further comprises:
a flexible substrate;
a flexible electronic circuit supported by the flexible substrate, wherein the flexible electronic circuit comprises said pressure sensor and said wireless communication system; and
a flexible superstrate layer encapsulating at least a portion of the flexible electronic circuit, the flexible substrate, or both the flexible electronic circuit and the flexible substrate.

23. The system of claim 22, wherein said flexible substrate and said flexible superstrate layer independently are characterized by an average modulus less than or equal to 1 MPa.

24. The system of claim 4, wherein said at least one flexible wireless device is a stretchable device.

25. The system of claim 24, wherein said stretchable device is capable of undergoing elongation or compression to an extent of at least a factor of 1.2 without system degradation or mechanical failure.

26. The system of claim 25, wherein said flexible wireless device of said stretchable device further comprises one or more stretchable filamentary electrical interconnects.

27. The system of claim 26, wherein said stretchable filamentary electrical interconnects have a serpentine, bent, folded, wavy or curved geometry.

28. The system of claim 4, wherein said pressure sensor is a capacitance pressure sensor, a piezoresistive pressure sensor or a combination thereof.

29. The system of claim 4, wherein said pressure sensor measures instantaneous pressure, average pressure, cumulative pressure or any combination of these.

30. The system of claim 4, wherein said pressure sensor measures the inherent extensibility of the functional fabric.

31. The system of claim 4, wherein said flexible wireless device is capable of determining the static stiffness index of the functional fabric.

32. The system of claim 4, wherein said at least one flexible wireless device further comprises one or more sensors selected from the group consisting of: a temperature sensor; a bioimpedence sensor; a radius of curvature sensor, an accelerometer, a heart rate sensor, a blood flow sensor, an electrocardiography sensor, an electromyography sensor, an electroencephalography sensor, an electrophysiological sensors, a moisture sensor, a humidity sensor, a transcutaneous oxygen sensor, a local blood flow sensor, and a local redness sensor.

33. The system of claim 4, wherein said at least one flexible wireless device further comprises a temperature sensor for measuring local temperature.

34. The system of claim 33, wherein said flexible wireless device uses said local temperature to provide a pressure measurement normalized by an in situ temperature.

35. The system of claim 4, wherein said at least one flexible wireless device further comprises an accelerometer for monitoring the motion or movement of a subject or detecting a fall of the subject.

36. The system of claim 4, wherein said at least one flexible wireless device further comprises a wound healing sensor.

37. The system of claim 36, wherein said wound healing sensor is for monitoring transcutaneous oxygen, local blood flow, local redness, local temperature, ultraviolet radiation, or any combination of them.

38. The system of claim 4, wherein said at least one flexible wireless device further comprises a light emitting diode.

39. The system of claim 38, wherein said light emitting diode is activated when said pressure sensor senses a pressure below a minimum compression threshold.

40-48. (canceled)

49. The system of claim 4, further comprising at least three wireless flexible devices.

50. The system of claim 49, wherein each of said wireless flexible devices independently comprise a sensor and a near-field communication system.

51. The system of claim 50, wherein said wireless flexible devices are positioned within said functional fabric such that when the covering is worn by a patient the wireless flexible devices contact said patient's gastrocnemius muscle, medial ankle and upper anterolateral thigh.

52. The system of claim 4, wherein said at least one flexible wireless device has an average thickness of less than or equal to 10 mm.

53. The system of claim 4, wherein said at least one flexible wireless device has an average thickness of less than or equal to 5 mm.

54. The system of claim 4, wherein said at least one flexible wireless device has a lateral area footprint less than or equal to 20,000 mm².

55. The system of claim 4, wherein said system further comprises one or more encapsulating layers.

56. The system of claim 55, wherein said flexible wireless device is entirely encapsulated by said one or more encapsulating layers.

57. The system of claim 55, wherein each of said encapsulating layers independently have a thickness less than or equal to 5 mm.

58. The system of claim 55, wherein said one or more encapsulating layers independently have a geometry without hard edges or concerns that provides for good adherence to the skin and reduced contact stresses.

59. The system of claim 55, wherein said one or more encapsulating layers independently have a geometry characterized by one or more curved surfaces.

60. The system of claim 55, wherein said one or more encapsulating layers independently have a dome shaped geometry.

61. The system of claim 60, wherein said dome shaped geometry is characterized by a fillet radius greater than or equal to 0.25 mm.

62. The system of claim 55, wherein said pressure sensor of said flexible wireless device is encapsulated by a lower encapsulating layer and an upper encapsulating layer.

63. The system of claim 62, wherein said pressure sensor is a piezoelectric sensor, capacitive sensor, a liquid metal sensor, a strain gauge sensor or an iontronic sensor.

64. The system of claim 62, wherein said an upper encapsulating layer has a thickness at least 1.5 times greater than a thickness of the lower encapsulating layer.

65. The system of claim 62, wherein said an upper encapsulating layer has a length at least 1.5 times less than a length of the lower encapsulating layer.

66. The system of claim 62, wherein said lower encapsulating layer has a geometry characterized by a thickness at least 8 times smaller than at least one lateral dimension.

67. The system of claim 62, wherein said lower encapsulating layer has a geometry characterized by a thickness at least 8 times smaller than a width of said lower encapsulating layer.

68. The system of claim 55, wherein said one or more encapsulating layer is polyimide, polydimethylsiloxane (PDMS), polyurethane, polystyrene, polymethyl methacrylate (PMMA) or polycarbonate.

69. The system of claim 4, wherein said wireless communication system is selected from the group consisting of: a transmitter, a receiver, a transceiver, an antenna, and a near field communication device.

70. The system of claim 4, wherein said wireless communication system is one or more near field coils.

71. The system of claim 70, wherein said one or more near field coils independently have a diameter selected from the range of 500 microns to 20 millimeters.

72. The system of claim 70, wherein said one or more near field coils independently has an average thickness selected from the range of 1 micron to 5 millimeters.

73. The system of claim 70, wherein said one or more near field coils independently has a geometry selected from the group consisting of: an annulus and an elliptical annulus.

74. The system of claim 4, wherein said wireless communication system provides Bluetooth wireless communication.

75. The system of claim 4, wherein said wireless communication system provides one-way or two-way wireless communication with an external device.

76. The system of claim 75, wherein said external device is a computer, a phone, a smartphone, a tablet or a diagnostic machine.

77. The system of claim 4, wherein said flexible wireless device further comprises a wireless energy harvester.

78. The system of claim 77, wherein said wireless energy harvester has an area antenna.

79. The system of claim 78, wherein said area antenna has a length that is greater than or equal to 100 cm.

80. The system of claim 77, wherein said wireless energy harvester provides a power delivery of greater than or equal to 1×10⁴ mW/cm².

81. The system of claim 4, wherein said flexible wireless device further comprises one or more battery.

82. The system of claim 4, wherein said flexible wireless device does not include a battery.

83. The system of claim 4, wherein said system further comprises a processor to provide a real-time metric.

84. The system of claim 83, wherein said processor is on-board with the flexible wireless device or is positioned in an external device that is located at a distance from the wireless device and in wireless communication with the wireless communication system.

85. The system of claim 4, wherein said flexible wireless device continuously monitors and generates a real-time metric.

86. The system of claim 4, wherein said flexible wireless device further comprises one or more of a vibratory motor, an electrode, a light emitter, or a thermal actuator.

87. The system of claim 4, comprising a plurality of spatially distributed flexible wireless devices.

88. The system of claim 87, wherein the plurality of spatially distributed flexible wireless devices are configured to provide an average output parameter and/or a spatial distribution map of an output parameter.

89. The system of claim 88, wherein said output parameter and/or spatial distribution map is time-varying.

90. The system of claim 87, that is a multiplexed system configured to provide an output for a plurality of output parameters.

91. The system of claim 87 configured to wirelessly communicate output parameters from the system for remote monitoring.

92. The system of claim 87, further comprising one or more actuators connected to the garment, wherein the actuators are configured to receive an input from a user, a medical professional or a feedback input based on one or more sensor outputs from one or more of the sensors.

93. A method of measuring an interface pressure of said functional fabric on a region of a body of a subject using the systems of claim 4.

94. The method of claim 93 wherein said interface pressure is an instantaneous pressure, an average pressure or a cumulative pressure.

95. A method of measuring a pressure and a limb volume upon administration of the functional fabric on a region of a body of a subject using the systems of claim 4.

96. A method of determining a limb volume response upon administration of the functional fabric on a region of a body of a subject using the systems of claim 4.

97. A method of treating edema, venous status ulcers, chronic venous insufficiency, deep vein thrombosis, lipodermatosclerosis, lymphedema or any combination of these using the systems of claim 4.

98. A method of assessing fabric failure of said functional fabric using said system of claim 4.

99. A method of monitoring patient adherence of said functional fabric using said system of claim 4.

100. A method of providing compression therapy to a subject comprising:
providing a system of claim 4; and
administering said system to a limb of the patent.

101. The method of claim 100, wherein said compression therapy is for treatment of venous status ulcers, chronic venous insufficiency, deep vein thrombosis, lipodermatosclerosis, lymphedema or a combination thereof.

102. The method of claim 100, wherein placement of said system in distinct anatomical locations provides an estimate of a global therapeutic pressure.

103-110. (canceled)
